(19) United States
(12) Patent Application Publication
(10) Pub. No.: US 2014/0194505 A1
(43) Pub. Date: Jul. 10, 2014

(54) Neuraminidase Inhibitors

(71) Applicant: SineVir Therapeutics LLC, Ann Arbor, MI (US)

(72) Inventors: John Hilfinger, Ann Arbor, MI (US); Wei Shen, Dearborn, MI (US)

(73) Assignee: SineVir Therapeutics LLC, Ann Arbor, MI (US)

(21) Appl. No.: 14/177,713

(22) Filed: Feb. 11, 2014

Related U.S. Application Data
(63) Continuation of application No. 13/638,686, filed on Oct. 1, 2012.
(60) Provisional application No. 61/320,454, filed on Apr. 2, 2010.

(51) Int. Cl. C07D 309/28; C07C 233/41; C07C 279/16

(52) U.S. Cl. CPC C07D 309/28; C07C 279/16; C07C 233/41
USPC 514/459; 560/125; 514/547; 549/424; 560/122

(57) ABSTRACT

Disclosed are neuraminidase inhibitor compounds and pharmaceutical compositions with improved bioavailability and/or improved efficacy and methods of treating influenza using the compounds and pharmaceuticals compositions.

### CROSS-REFERENCE TO RELATED APPLICATIONS

This application is a continuation U.S. Ser. No. 13/638,686, filed on Oct. 1, 2012, which is a national stage application under 35 U.S.C. 371 of PCT/US2011/031109, filed Apr. 4, 2011, which claims priority to U.S. Provisional Application Ser. No. 61/320,454 filed Apr. 2, 2010. The entire content of all of the above referenced applications are incorporated herein by reference.

### FIELD

Neuraminidase inhibitors for use as pharmaceutical agents.

### BACKGROUND

Numerous potentially effective therapeutic agents often exhibit poor bio-pharmaceutical properties such as low solubility or low bioavailability which can preclude the effective oral use of a potential therapeutic agent. Some drugs having low bioavailability are effective when administered by non-oral route, e.g., intravenously. However, oral administration of pharmaceutical agents is generally the preferred route of administration for reasons such as ease of administration, cost, and patient compliance. Although much effort is being applied to increasing the bioavailability of drugs with poor bioavailability, including marketed drugs and drugs still in development, this effort has been met with limited success.

Thus, there exists a need for enhancing the bioavailability of therapeutic drugs.

### SUMMARY

Provided are compounds of the general formula (I)

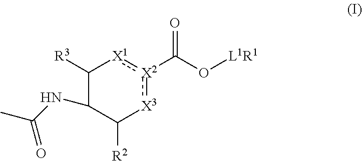

wherein: L¹ is —(CR^{o}R^{o})ₘC(R⁴)₂(CR^{o}R^{o})ₙO(CR^{o}R^{o})—;
R¹ is —C(O)(CR^{o}R^{o})ᵣC(R^{o}R′)(CR^{o}R^{o})ₛNH₂, —C(O)(CR^{o}R^{o})ᵣC(R^{o}R′)(CR^{o}R^{o})ₛN(H)C(O)(CR^{o}R^{o})_{w}C(R^{o}R″)(CR^{o}R^{o})ₓNH₂, or —C(O)(CR^{o}R^{o})C(R^{o}R′)(CR^{o}R^{o})ₛN(H)C(O)(CR^{o}R^{o})_{w}C(R^{o}R″)(CR^{o}R^{o})ₓN(H)C(O)(CR^{o}R^{o})_{y}C(R^{o}R′″)(CR^{o}R^{o})NH₂;
each occurrence of m, n, o, r, s, w, x, y, or z is independently zero, one, or two;
each occurrence of R^{o} is independently H, optionally substituted alkyl, optionally, substituted cycloalkyl, optionally substituted aryl, or optionally substituted heteroaryl;

### R² is NH₂ or —NHC(NH₂)NH;

### R³ is H, —OR*, or —CHR*R**;

R′, R″ and R′″ are each independently an amino acid side chain;
each occurrence of R⁴ is independently hydrogen or an optionally substituted group selected from a C₁-C₆ alkyl group, a 3-7 membered saturated, partially saturated or fully unsaturated monocyclic ring having 0-3 heteroatoms independently selected from nitrogen or oxygen or two occurrences of R⁴ are taken together with the atom(s) to which they are bound to form an optionally substituted 3-7 membered ring, wherein if one occurrence of R⁴ is H, then the other occurrence of R⁴ is not H or —CH₃;
R* and R** are independently, H, OH, —OR⁵, or optionally substituted C₁-C₁₂ alkyl;
R⁵ is optionally substituted C₁-C₆ alkyl, or —C(O)NR^{o}R^{o};
X¹ is O or CH wherein if X¹ is 0, then there is a single bond between X¹ and X² and a double bond between
X² and X³; and wherein X¹ is CH then there is a double bond between X¹ and X² and a single bond between X² and X³;

### X² is C; and

### X³ is CH or CH₂;

or a pharmaceutically acceptable salt thereof.

Also provided are compounds of the general formula (IV):

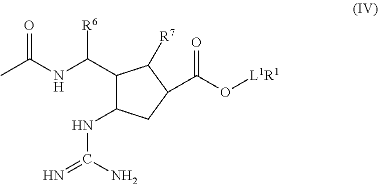

wherein: L¹ is —(CR^{o}R^{o})ₘC(R⁴)₂(CR^{o}R^{o})ₙO(CR^{o}R^{o})ₒ—;
R¹ is —C(O)(CR^{o}R^{o})ᵣC(R^{o}R′)(CR^{o}R^{o})ₛNH₂, —C(O)(CR^{o}R^{o})ᵣC(R^{o}R′)(CR^{o}R^{o})ₛN(H)C(O)(CR^{o}R^{o})_{w}C(R^{o}R″)(CR^{o}R^{o})ₓNH₂, or —C(O)(CR^{o}R^{o})ᵣC(R^{o}R′)(CR^{o}R^{o})ₛN(H)C(O)(CR^{o}R^{o})_{w}C(R^{o}R″)(CR^{o}R^{o})ₓN(H)C(O)(CR^{o}R^{o})_{y}C(R^{o}R′″)(CR^{o}R^{o})_{z}NH₂;
each occurrence of m, n, o, r, s, w, x, y, or z is independently zero, one, or two;
each occurrence of R^{o} is independently H, alkyl, substituted alkyl, cycloalkyl, substituted cycloalkyl, aryl, substituted aryl, heteroaryl and substituted heteroaryl;
each occurrence of R⁴ is independently hydrogen or an optionally substituted group selected from a C₁-C₆ alkyl group, a 3-7 membered saturated, partially saturated or fully unsaturated monocyclic ring having 0-3 heteroatoms independently selected from nitrogen or oxygen or two occurrences of R⁴ are taken together with the atom(s) to which they are bound to form an optionally substituted 3-7 membered ring, wherein if one occurrence of R⁴ is H, then the other occurrence of R⁴ is not H or CH₃;
R⁵ is optionally substituted C₁-C₄ alkyl, —C(O)NR^{o}R^{o};
R⁶ is C₁-C₁₀ alkyl; and
R⁷ is —OH, —OR⁵, C₁-C₆ alkyl or —NR^{o}R^{o};
or a pharmaceutically acceptable salt thereof.

Also provided are pharmaceutical compositions of formulas (I) and (IV) and methods of treatment of viral infections using the compounds of the present disclosure.

### BRIEF DESCRIPTION OF THE DRAWINGS

FIG. 1 shows a comparison of the GOC plasma levels after oral administration of GOC-Isp-Val or GOC and IV administration of GOC to fed animals.

FIG. 2 are graphs show the extent of weight loss, of mice infected with influenza A virus after administration of GOC, analogs of GOC, and oseltamivir.

FIG. 3 shows is a graph showing a comparison of Zanamivir plasma levels after oral administration of Zanamivir or ZAN-Isp-Val.

### DETAILED DESCRIPTION

The compounds according to the present disclosure are analogs of neuraminidase inhibitors including, but not limited to, zanamivir, oseltamivir, peramivir, laninamivir (R-125489), and a prodrug of laninamivir (Daiichi Sankyo Co. Ltd. code name CS-8958), having increased oral bioavailability. As used herein the term “base compound” refers to compounds which do not include a modification at the carboxyl group. For example, base compounds include but are not limited to, zanamivir, oseltamivir, peramivir, laninamivir (R-125489), and a prodrug of laninamivir (Daiichi Sankyo Co. Ltd. code name CS-8958).

The neuraminidase inhibitor analogs according to the present disclosure may be cleaved in vivo by endogenous enzymatic mechanisms. For example, the analogs may be hydrolyzed by endogenous hydrolytic enzymes, including but not limited to, valacyclovirase, influenza virus protease or a human cytomegalovirus (HCMV) protease.

With respect to the formulae used herein, a parenthetical group is bonded to the immediately preceding non-hydrogen atom and not to the immediately succeeding non-hydrogen atom. This convention as to the use of parenthetical groups does not apply when the parenthetical group is immediately succeeded by a subscript of m, n, o, r, s, w, x, y, or z.

The terms “alkyl” and “alkyl group” are used interchangeably and mean a linear, branched, saturated or unsaturated carbon chain having 1 to 20 carbon atoms. The number of carbon atoms can be expressed, for example, “C₁-C₅ alkyl” which means that the alkyl group has one to five carbon atoms. Examples of such groups include methyl, ethyl, n-propyl, isopropyl, n-butyl, isobutyl, sec-butyl, tert-butyl, pentyl, 1,4-dienyl, but-1-enyl and the like.

An alkyl group may be optionally substituted with OH, alkyl, phenyl, benzyl, amide, amine, imine, carbamide, aziridine, hydrizine, nitrile, isocyanate, ketone, aldehyde, ester, ether, carboxylic acid, carboxylate salt peroxide, epoxide, ketal, acetal thioether, thioester, disulfide, sulfone, thioamide, thio, thione, sulfoxide, isothiocyanate, sulfonamide or halogen.

The terms “cycloalkyl” and “cycloalkyl group” are used interchangeably and mean a saturated mono-ring carbocycle with three to seven atoms on the ring. Examples of such groups include cyclopropyl, cyclobutyl, cyclopentyl, cyclohexyl, and cycloheptyl.

A cycloalkyl group may be optionally substituted with OH, alkyl, phenyl, benzyl, amide, amine, imine, carbamide, aziridine, hydrizine, nitrile, isocyanate, ketone, aldehyde, ester, ether, carboxylic acid, carboxylate salt peroxide, epoxide, ketal, acetal thioether, thioester, disulfide, sulfone, thioamide, thio, thione, sulfoxide, isothiocyanate, sulfonamide or halogen.

The term “amide” means —C(O)NR^{o}R^{o}— or —NR^{o}R^{o}C(O)— wherein each occurrence of R^{o} is independently selected from H, alkyl, substituted alkyl, cycloalkyl, substituted cycloalkyl, aryl, substituted aryl, heteroaryl and substituted heteroaryl.

The term amine means —NR^{o}R^{o} wherein each occurrence of R^{o} is independently selected from H, alkyl, substituted alkyl, cycloalkyl, substituted cycloalkyl, heteroaryl and substituted heteroaryl.

The terms “aryl” and “aryl group” are used interchangeably and mean an unsaturated 5 to 9 membered carbocyclic ring or a polycyclic (e.g., bicyclic) ring in which two or more monocyclic aryl rings are fused together to form a conjugated ring system. Typical rings include phenyl, napthyl, phenanthryl, anthracenyl, toluenyl, anilinyl, chrysenyl, naphthacenyl, pyrenyl, purinyl, adeninyl, guaninyl, hypoxanthinyl, xanthinyl, theobrominyl, caffeinyl, and isoguaninyl.

An aryl group may be optionally substituted with a group selected from alkyl, OH, nitro, amide, amine, imine, aryl, heteroaryl, carbamide, aziridine, hydrazine, nitrile, isocyanate, ketone, aldehyde, ester, ether, carboxylic acid, carboxylate salt, peroxide, epoxide, ketal, acetal, thioether, thioester, disulfide, sulfone, thioamide, thiol, thione, sulfoxide, isothiocyanate, sulfoamide, or halogen.

The terms “heteroaryl” and “heteroaryl group” are used interchangeably and mean an unsaturated five to nine membered cyclic ring incorporating one or more heteroatoms independently selected from N and O.

A heteroaryl group may be optionally substituted with a group selected from alkyl, OH, nitro, amide, amine, imine, aryl, heteroaryl, carbamide, aziridine, hydrazine, nitrile, isocyanate, ketone, aldehyde, ester, ether, carboxylic acid, carboxylate salt, peroxide, epoxide, ketal, acetal, thioether, thioester, disulfide, sulfone, thioamide, thiol, thione, sulfoxide, isothiocyanate, sulfoamide, or halogen.

Any reference in the claims to “optionally substituted” includes “unsubstituted” and “substituted”. Where a group is designated as “unsubstituted” then that group is not substituted.

The term “analog” in the context of this application is interchangeable with “neuraminidase inhibitor analog”.

“GOC” means 4-guanidinio oseltamivir carboxylate.

“MOM” means methoxymethyl.

As used herein, a “therapeutically effective amount” is defined to include an amount necessary to delay the onset of, inhibit the progress of, relieve the symptoms of, or reverse a condition being treated.

Naturally occurring or non-naturally occurring amino acids are used to prepare the analogs according to the present disclosure. Suitable amino acids include, but are not limited to, standard amino acids such as valine, leucine, isoleucine, methionine, phenylalanine, asparagine, glutamic acid, glutamine, histidine, lysine, arginine, aspartic acid, serine, threonine, tyrosine, tryptophan, cysteine, and proline. The use of L-amino acids and D-amino acids are contemplated. L-amino acids are more often kinetically faster cleavage substrates for autologous subject enzymes. Incorporation of D-amino acids into a compound may stabilize it allowing more time for the compound to be absorbed. Depending on the compound and the application, one of skill in the art would understand that the compounds could be tailored to address a specific situation. Also, naturally occurring, non-standard amino acids are operative in the compositions and methods of the invention. For example, amino acids further include 4-hydroxyproline, γ-carboxyglutamic acid, selenocysteine, 6-N-methyllysine, ε-N,N,N-trimethyllysine, 3-methylhistidine, O-phosphoserine, N-acetylserine, 5-hydroxylysine, ε-N-acetyllysine, ω-N-methylarginine, citrulline, ornithine, azaserine, homocysteine, and β-cyanoalanine. Non-naturally occurring amino acids include, but are not limited to, phenyl glycine, meta-tyrosine, para-amino phenylalanine, 3-(3-pyridyl)-L-alanine, 4-(trifluoromethyl)-D-phenylalanine, and the like. Also, the use of β and γ amino acids is contemplated. For example β-valine, γ-valine, γ-aminobutyric acid and the like.

It is appreciated that analogs according to the present disclosure are useful to treat a variety of diseases responsive to neuraminidase inhibition. In particular, methods of treating viral infection using analogs of neuraminidase inhibitors are provided by the present disclosure. Illustratively, infection by influenza A virus and/or influenza B virus are treated using analogs of neuraminidase inhibitors.

In some embodiments, the analogs of the present disclosure are formulated for administration to humans. However, it is appreciated that use of the analogs may be indicated for administration to a non-human organism, for example, of the rodent, porcine, bovine, equine, avian, canine, or feline families wherein the organism is susceptible to influenza.

Provided by the present disclosure are compounds of the general formula (I):

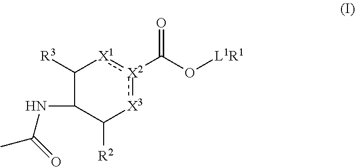

wherein: L¹ is —(CR^{o}R^{o})ₘC(R⁴)₂(CR^{o}R^{o})ₙO(CR^{o}R^{o})ₒ—;
R¹ is —C(O)(CR^{o}R^{o})ᵣC(R^{o}R′)(CR^{o}R^{o})ₛNH₂;
—C(O)(CR^{o}R^{o})ᵣC(R^{o}R′)(CR^{o}R^{o})ₛN(H)C(O)(CR^{o}R^{o})_{w}C(R^{o}R″)(CR^{o}R^{o})ₓNH₂, or —C(O)(CR^{o}R^{o})ᵣC(R^{o}R′)(CR^{o}R^{o})ₛN(H)C(O)(CR^{o}R^{o})_{w}C(R^{o}R″)(CR^{o}R^{o})ₓN(H)C(O)(CR^{o}R^{o})_{y}C(R^{o}R″″)(CR^{o}R^{o})_{z}NH₂;
each occurrence of m, n, o, r, s, w, x, y, or z is independently zero, one, or two;
each occurrence of R^{o} is independently H, alkyl, substituted alkyl, cycloalkyl, substituted cycloalkyl, aryl, substituted aryl, heteroaryl and substituted heteroaryl;

### R² is NH₂ or —NHC(NH₂)NH,

### R³ is H, —OR*, or —CHR*R**;

R′, R″ and R′″ are each independently an amino acid side chain;
each occurrence of R⁴ is independently hydrogen or an optionally substituted group selected from a C₁-C₆ alkyl group, a 3-7 membered saturated, partially saturated or fully unsaturated monocyclic ring having 0-3 heteroatoms independently selected from nitrogen or oxygen or two occurrences of R⁴ are taken together with the atom(s) to which they are bound to form an optionally substituted 3-7 membered ring, wherein if one occurrence of R⁴ is H, then the other occurrence of R⁴ is not H or CH₃;

R* and R** are independently, H, OH, —OR⁵, or C₁-C₅ alkyl optionally substituted with —OH, —OR⁵, or —OC(O)(C₁-C₅ alkyl);

R⁵ is optionally substituted C₁-C₄ alkyl, C(O)NR^{o}R^{o};
X¹ is O or CH wherein if X¹ is O, then there is a single bond between X¹ and X² and a double bond between X² and X³; and wherein X¹ is CH then there is a double bond between X¹ and X² and a single bond between X² and X³;

### X² is C; and

### X³ is CH or CH₂;

or a pharmaceutically acceptable salt thereof.

### Oseltamivir Carboxylate and 4-Guanidinio Oseltamivir Carboxylate Analogs

Oseltamivir carboxylate is a potent inhibitor of influenza virus neuraminidase (IC₅₀˜2 nM). The guanidine analog of oseltamivir carboxylate (4-guanidinio oseltamivir carboxylate) is an approximately 2-fold more potent inhibitor in vitro (IC₅₀=0.9 nM) but is 10 times more potent in tissue culture of influenza virus replication. However, both oseltamivir carboxylate and the guanidine analog of oseltamivir carboxylate are poorly bioavailable (˜4.0%). Oseltamivir (Tamiflu), the ethyl ester analog of oseltamivir carboxylate, is administered orally. However, the ethyl ester prodrug of the more potent guanidine analog, does not exhibit enhancement in oral bioavailability (˜2%).

Analogs of oseltamivir carboxylate and 4-guanidinio oseltamivir carboxylate are provided according to the present disclosure. For reference, the base compounds are shown below:

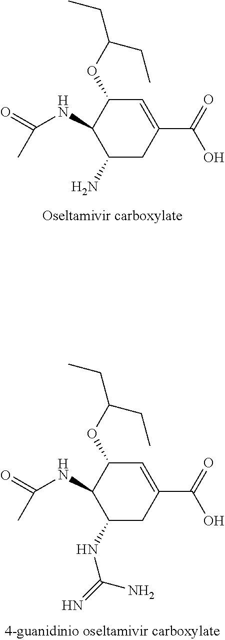 [SMILES: CCC(CC)O[C@@H]1C=C(C(=O)O)C[C@H](N)[C@H]1NC(C)=O ; CCC(CC)O[C@@H]1C=C(C(=O)O)C[C@H](NC(=N)N)[C@H]1NC(C)=O]

Analogs of oseltamivir carboxylate and 4-guanidinio oseltamivir carboxylate according to embodiments of the present disclosure are represented by formula (ID:

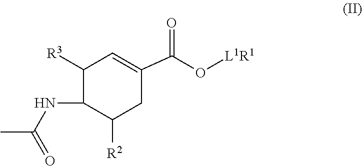

wherein: L¹, R¹, R² and R³ are defined as in formula (I).

### Analogs of Zanamivir

Zanamivir has been shown to be a potent inhibitor of both influenza A and influenza B and of emerging resistant strains. However, the low absolute oral availability of zanamivir, about 2%, precludes oral administration. Analogs of zanamivir are provided in the present disclosure include are modified at the carboxyl functional groups of the base compound. For reference, the base compound zanamivir is shown below:

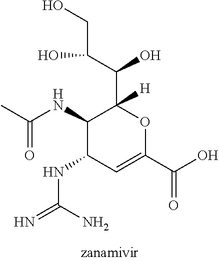 [SMILES: CC(=O)N[C@H]1[C@H]([C@H](O)[C@H](O)CO)OC(C(=O)O)=C[C@@H]1NC(=N)N]

### Analogs of Laninamivir (R-125489), and CS-8958

CS-8958, a prodrug of Laninamivir (R-125489), is currently marketed in Japan. Laninamivir is administered by inhalation and is reported to show long-acting anti-virus activity toward both influenza A and influenza B. Analogs of Laninamivir and CS-8958 provided in the present disclosure are modified at the carboxyl functional groups of the base compound. For reference, the base structures are shown below:

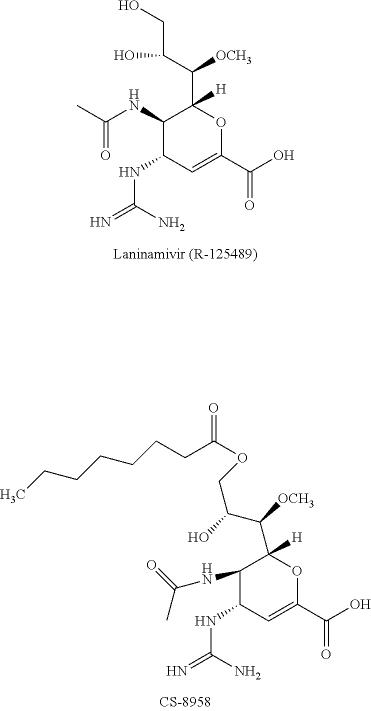 [SMILES: CO[C@@H]([C@@H]1OC(C(=O)O)=C[C@H](NC(=N)N)[C@H]1NC(C)=O)[C@H](O)CO ; CCCCCCCC(=O)OC[C@@H](O)[C@@H](OC)[C@@H]1OC(C(=O)O)=C[C@H](NC(=N)N)[C@H]1NC(C)=O]

Analogs of Zanamivir, CS-8958 and Laninamivir according to embodiments of the present disclosure are represented by formula (III):

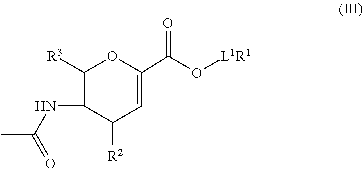

wherein: L¹, R¹, R² and R³ are defined as in formula (I).

Illustrative embodiments of the variables R², R³, R*, R** and R⁵ of the compounds of formula (1), formula (II) or formula (III) are described below.

In certain embodiments R² is —NHC(NH₂)NH. In another embodiment R² is NH₂.

In one embodiment R³ is H. In another embodiment R³ is —OR*. In yet another embodiment R³ is —CHR*R**. In another embodiment R³ is —CH(OR⁵)CH(OR⁵)CH₂(OR⁵) where each occurrence of R⁵ is independently H or optionally substituted C₁-C₆ alkyl. In yet another embodiment R³ is —OCH(CH₂CH₃)₂. In another embodiment, R³ is —CH(OCH₃)CH₂(OH)CH₂OC(O)(CH₂)₆CH₃. In one embodiment R³ is —CH(OH)CH(OH)CH₂(OH).

In some embodiments R* is H, or R* is —OH, or R* is —OR⁵. In other embodiments R* is optionally substituted C₁-C₁₂ alkyl, or R* is unsubstituted C₁-C₁₂ alkyl. In another embodiment R* is optionally substituted C₁-C₈ alkyl, or R* is unsubstituted C₁-C₈ alkyl. In yet another embodiment R* is optionally substituted C₁-C₆ alkyl, or R* is unsubstituted C₁-C₆ alkyl. In one embodiment R** is H, or R** is —OH, or R** is —OR⁵. In another embodiment R** is optionally substituted C₁-C₁₂ alkyl, or R** is unsubstituted C₁-C₁₂ alkyl. In another embodiment R** is optionally substituted C₁-C₈ alkyl, or R** is unsubstituted C₁-C₈ alkyl. In yet another embodiment R** is optionally substituted C₁-C₆ alkyl, or R** is unsubstituted C₁-C₆ alkyl. In certain embodiments R* is H and R** is H, or R* is H and R** is —OH, or R* is H and R** is —OR⁵, or R* is H and R** is optionally substituted C₁-C₁₂ alkyl, or R* is H and R** is unsubstituted C₁-C₁₂ alkyl or R* is H and R** is optionally substituted C₁-C₈ alkyl, or R* is H and R** is unsubstituted C₁-C₈ alkyl or R* is H and R** is optionally substituted C₁-C₆ alkyl, or R* is H and R** is unsubstituted C₁-C₆ alkyl. In certain embodiments R* is —OH and R** is H, or R* is —OH and R** is —OH, or R* is —OH and R** is —OR⁵, or R* is —OH and R** is optionally substituted C₁-C₁₂ alkyl, or R* is —OH and R** is unsubstituted C₁-C₁₂ alkyl or R* is —OH and R** is optionally substituted C₁-C₈ alkyl, or R* is —OH and R** is unsubstituted C₁-C₈ alkyl or R* is —OH and R** is optionally substituted C₁-C₆ alkyl, or R* is —OH and R** is unsubstituted C₁-C₆ alkyl. In certain embodiments R* is —OR⁵ and R** is H, or R* is —OR⁵ and R** is —OH, or R* is —OR⁵ and R** is —OR⁵, or R* is —OR⁵ and R** is optionally substituted C₁-C₁₂ alkyl, or R* is —OR⁵ and R** is unsubstituted C₁-C₁₂ alkyl or R* is —OR⁵ and R** is optionally substituted C₁-C₈ alkyl, or R* is —OR⁵ and R** is unsubstituted C₁-C₈ alkyl or R* is —OR⁵ and R** is optionally substituted C₁-C₆ alkyl, or R* is —OR⁵ and R** is unsubstituted C₁-C₆ alkyl.

### Peramivir Analogs

Peramivir is a cyclopentane neuraminidase inhibitor that exhibits in vitro and in vivo activity against various influenza A and B viruses including the highly pathogenic H5N1 viruses. Peramivir has demonstrated a good safety profile when tested in mice, rats, primates and dogs, following oral, intravenous and intramuscular administration. However peramivir failed to achieve significant clinical effects in phase 2 and phase 3 clinical trials owing to its low oral bioavailability 3%).

Peramivir analogs having enhanced bioavailability compared with the base compound are provided according to embodiments of the present disclosure. For reference, the base compound peramivir is shown below:

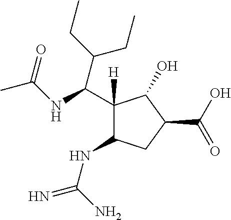

Analogs of peramivir according to embodiments of the present disclosure are represented by formula (IV):

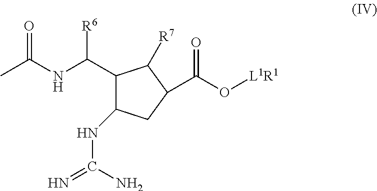

wherein: L¹ is —(CR^{o}R^{o})ₘC(R⁴)₂(CR^{o}R^{o})ₙO(CR^{o}R^{o})ₒ—;
—R¹ is —C(O)(CR^{o}R^{o})ᵣCH(R′)(CR^{o}R^{o})ₛNH₂, —C(O)(CR^{o}R^{o})ᵣCH(R′)(CR^{o}R^{o})ₛN(H)C(O)(CR^{o}R^{o})_{w}CH(R″)(CR^{o}R^{o})ₓNH₂, or —C(O)(CR^{o}R^{o})ᵣCH(R′)(CR^{o}R^{o})ₛN(H)C(O)(CR^{o}R^{o})_{w}CH(R″)(CR^{o}R^{o})ₓN(H)C(O)(CR^{o}R^{o})_{y}CH(R″)(CR^{o}R^{o})_{z}NH₂;
each occurrence of m, n, o, r, s, w, x, y, or z is independently zero, one, or two;
each occurrence of R^{o} is independently alkyl, substituted alkyl, cycloalkyl, substituted cycloalkyl, aryl, substituted aryl, heteroaryl and substituted heteroaryl;
each occurrence of R⁴ is independently hydrogen or an optionally substituted group selected from a C₁-C₆ alkyl group, a 3-7 membered saturated, partially saturated or fully unsaturated monocyclic ring having 0-3 heteroatoms independently selected from nitrogen or oxygen or two occurrences of R⁴ are taken together with the atom(s) to which they are bound to form an optionally substituted 3-7 membered ring, wherein if one occurrence of R⁴ is H, then the other occurrence of R⁴ is not H or CH₃;
R⁵ is optionally substituted C₁-C₄ alkyl, —C(O)NR^{o}R^{o};
R⁶ is C₁-C₁₀ alkyl; and
R⁷ is OH, —OR⁵, C₁-C₆ alkyl or —NR^{o}R^{o};
or pharmaceutically acceptable salts thereof.

In certain embodiments of formula (IV), R⁶ is —CH₃, —CH₂CH₃, —CH₂CH₂CH₃, —CH(CH₃)₂, —CH₂CH₂CH₂CH₃, —CH(CH₃)CH₂CH₃, —CH₂CH(CH₃)₂, —C(CH₃)₃, —CH₂(CH₂)₃CH₃, —CH(CH₃)(CH₂)₂CH₃, —CH(CH₃)CH(CH₃)₂, —C(CH₃)₂CH₂CH₃, —CH(CH₂CH₃)₂, —CH₂CH(CH₃)CH₂CH₃, —CH₂CH₂CH(CH₃)₂, —CH₂C(CH₃)₃, —CH₂(CH₂)₄CH₃, —CH(CH₃)(CH₂)₃CH₃, —CH₂CH(CH₃)(CH₂)₂CH₃, —CH₂CH₂CH(CH₃)CH₂CH₃, —CH₂ (CH₂)₂CH(CH₃)₂, —CH(CH₃)CH(CH₃)CH₂CH₃, —CH(CH₃)CH₂CH(CH₃)₂, —CH₂CH(CH₃)CH(CH₃)₂, —CH₂CH₂CH₂CH(CH₃)₂, —C(CH₃)₂CH₂CH₂CH₃, —C(CH₃)₂CH(CH₃)₂, —C(CH₂CH₃)₂CH₃, or —CH₂CH(CH₂CH₃)₂. In particular embodiments R⁶ is —C(CH₂CH₃)₂.

In certain embodiments of formula (IV), R⁷ is OH. In other embodiments R⁷ is —OR⁵. In yet other embodiments R⁷ is C₁-C₆ alkyl. In particular embodiments R⁷ is —CH₃, —CH₂CH₃, —CH₂CH₂CH₃, —CH(CH₃)₂, —CH₂CH₂CH₂CH₃, —CH(CH₃)CH₂CH₃, —CH₂CH(CH₃)₂, —C(CH₃)₃, —CH₂(CH₂)₃CH₃, —CH(CH₃)(CH₂)₂CH₃, —CH(CH₃)CH(CH₃)₂, —C(CH₃)₂CH₂CH₃, —CH(CH₂CH₃)₂, —CH₂CH(CH₃)CH₂CH₃, —CH₂CH₂CH(CH₃)₂, —CH₂C(CH₃)₃, —CH₂(CH₂)₄—CH₃, —CH(CH₃)(CH₂)₃CH₃, —CH₂CH(CH₃)(CH₂)₂CH₃, —CH₂CH₂CH(CH₃)CH₂CH₃, —CH₂ (CH₂)₂CH(CH₃)₂, —CH(CH₃)CH(CH₃)CH₂CH₃, —CH(CH₃)CH₂CH(CH₃)₂, —CH₂CH(CH₃)CH(CH₃)₂, —CH₂CH₂CH₂CH(CH₃)₂, —C(CH₃)₂CH₂CH₂CH₃, —C(CH₃)₂CH(CH₃)₂, —C(CH₂CH₃)₂CH₃, or —CH₂CH(CH₂CH₃)₂.

In still another embodiment R⁷ is —NR^{o}R^{o}. In various embodiments of R⁷ is —NR^{o}R^{o}, each occurrence of R^{o} is independently C₁-C₆ alkyl, substituted C₁-C₆ alkyl, optionally substituted C₃-C₇ cycloalkyl, optionally substituted C₅-C₉ aryl, or an optionally substituted 5-9 membered heteroaryl ring having from 0-3 heteroatoms independently selected from S, N and O. In some embodiments each occurrence of R^{o} is independently —CH₃, —CH₂CH₃, —CH₂CH₂CH₃, —CH(CH₃)₂, —CH₂CH₂CH₂CH₃, —CH(CH₃)CH₂CH₃, —CH₂CH(CH₃)₂, —C(CH₃)₃, —CH₂(CH₂)₃CH₃, —CH(CH₃)(CH₂)₂CH₃, —CH(CH₃)CH(CH₃)₂, —C(CH₃)₂CH₂CH₃, —CH(CH₂CH₃)₂, —CH₂CH(CH₃)CH₂CH₃, —CH₂CH₂CH(CH₃)₂, —CH₂C(CH₃)₃, —CH₂(CH₂)₄CH₃, —CH(CH₃)(CH₂)₃CH₃, —CH₂CH(CH₃)(CH₂)₂CH₃, —CH₂CH₂CH(CH₃)CH₂CH₃, —CH₂ (CH₂)₂CH(CH₃)₂, —CH(CH₃)CH(CH₃)CH₂CH₃, —CH(CH₃)CH₂CH(CH₃)₂, —CH₂CH(CH₃)CH(CH₃)₂, —CH₂CH₂CH₂CH(CH₃)₂, —C(CH₃)₂CH₂CH₂CH₃, —C(CH₃)₂CH(CH₃)₂, —C(CH₂CH₃)₂CH₃, or —CH₂CH(CH₂CH₃)₂.

Various embodiments of formula (I), formula (II), formula (III) and formula (IV) are exemplified below.

Each occurrence of m, n, o are independently zero, one, or two. For example m, n, and o may respectively be: 0, 0, 0; 0, 1, 0; 0, 2, 0; 0, 0, 1; 0, 0, 2; 0, 1, 1; 0, 2, 2; 0, 1, 2; 0, 2, 1; 1, 1, 0; 1, 2, 0; 1, 0, 1; 1, 0, 2; 1, 1, 1; 1, 2, 2; 1, 1, 2; 1, 2, 1; 2, 1, 0; 2, 2, 0; 2, 0, 1; 2, 0, 2; 2, 1, 1; 2, 1, 2; 2, 2, 1; or 2, 2, 2. Similarly each occurrence of r, s, w, x, y, and z are independently zero, one or two. It is to be understood that each and every combination of m, n, o, r, s, w, x, y, and z are contemplated as part of the present invention. In some embodiments m, n, o, r, s, w, x, y, and z are zero. In other embodiments m, n, o are 0 and one of r or s, is 1 and the other is 0. In other embodiments one of m, n, and o is 1 and the others are 0 and r, s, w, x, y, and z are 0. In another embodiment m+n+o+r+s+w+x+y+z=1. In another embodiment m+n+o+r+s+w+x+y+z=2. In another embodiment m+n+o=0 and r+s=1. In another embodiment m+n+o=0 and r+s=2. In another embodiment m+n+o=1 and r+s=0. In another embodiment m+n+o=1 and r+s=1.

In certain embodiments L¹ is —C(R⁴)₂O—. In other embodiments L¹ is —(CR^{o}R^{o})C(R⁴)₂O—. In yet other embodiments L¹ is —(CR^{o}R^{o})C(R⁴)₂(CR^{o}R^{o})O—. In other embodiments L¹ is —(CR^{o}R^{o})(CR^{o}R^{o)}—C(R⁴)₂—O—. In other embodiments L¹ is —C(R⁴)₂(CR^{o}R^{o})O(CR^{o}R^{o})—. In other embodiments L¹ is —(CR^{o}R^{o})—C(R⁴)₂(CR^{o}R^{o})O(CR^{o}R^{o})—. In yet other embodiments L¹ is —C(R⁴)₂O(CR^{o}R^{o})—(CR^{o}R^{o}).

In certain embodiments R′, R″ and R″ are each independently an amino acid side chain selected from H, —CH₃, —CH(CH₃)CH₂CH₃, —CH₂CH(CH₃)₂, —CH₂OH, —CH(CH₃)₂, —CH₂C(O)OH, —CH₂CH₂C(O)OH, —CH₂CH₂CH₂CH₂NH₂, —CH₂CH₂CH₂NHC(═NH)NH₂, —CH₂SH, —CH₂C(O)NH₂, —CH₂CH₂C(O)NH₂, —CH(OH)CH₃, —CH₂CH₂SCH₃, —CH₂CH(COOH)₂, —CH₂SeH, —CH₂CH₂CH₂CH₂NHCH₃, —CH₂CH₂CH₂CH₂N⁺(CH₃)₃, —CH₂OPO₃H₂, —CH₂CH₂CH(OH)CH₂NH₂, —CH₂CH₂CH₂CH₂NHC(O)CH₃, —CH₂CH₂CH₂NHC(═NH)NHCH₃, —CH₂CH₂CH₂NHC(O)NH₂, —CH₂CH₂CH₂NH₂, —CH₂OC(O)CHN₂, —CH₂CH₂SH, CH₂CN,

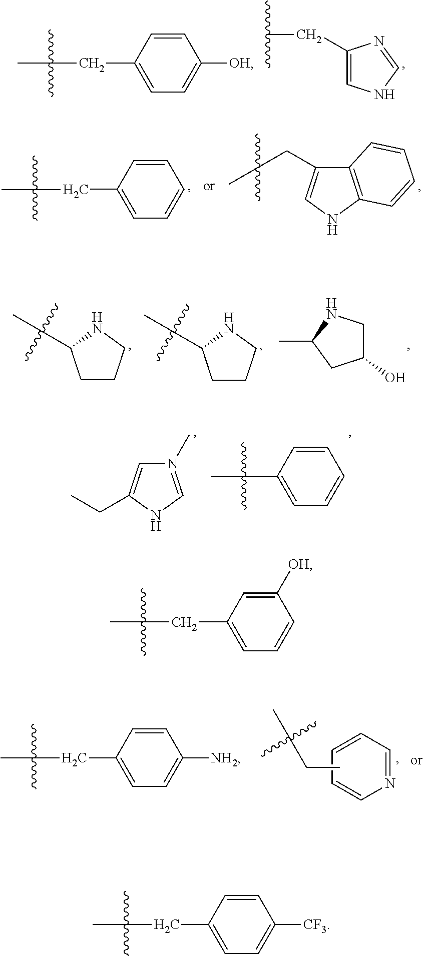

In other embodiments R′, R″ and R′″ are each independently H, —CH₃, CH(CH₃)CH₂CH₃, —CH₂CH(CH₃)₂, —CH(CH₃)₂, —CH₂OH, or —CH₂CH₂CH₂CH₂NH₂.

Tables A1 and A2 below provide illustrative examples of R⁴. Each occurrence of R⁴ is independently selected. Table A1 provides examples in which the two occurrences R⁴ do not form a ring. Table A2 provides examples in which the two occurrences of R⁴ are taken together with the atom(s) to which they are bound to form an optionally substituted 3-7 membered ring, wherein if one occurrence of R⁴ is H, then the other occurrence of R⁴ is not H or —CH₃. Table B1-B3 below provides examples of R¹.

TABLE A1

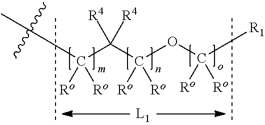

| No. | R⁴ |
|---|---|
| 1 | H |
| 2 | —CH₃ |
| 3 | —CH₂CH₃ |
| 4 | —CH₂CH₂CH₃ |
| 5 | —CH(CH₃)₂ |
| 6 | —CH₂CH₂CH₂CH₃ |
| 7 | —CH(CH₃)CH₂CH₃ |
| 8 | —CH₂CH(CH₃)₂ |
| 9 | —C(CH₃)₃ |
| 10 | —CH₂(CH₂)₃CH₃ |
| 11 | —CH(CH₃)(CH₂)₂CH₃ |
| 12 | —CH(CH₃)CH(CH₃)₂ |
| 13 | —C(CH₃)₂CH₂CH₃ |
| 14 | —CH(CH₂CH₃)₂ |
| 15 | —CH₂CH(CH₃)CH₂CH₃ |
| 16 | —CH₂CH₂CH(CH₃)₂ |
| 17 | —CH₂C(CH₃)₃ |
| 18 | —CH₂(CH₂)₄CH₃ |
| 19 | —CH(CH₃)(CH₂)₃CH₃ |
| 20 | —CH₂CH(CH₃)(CH₂)₂CH₃ |
| 21 | —CH₂CH₂CH(CH₃)CH₂CH₃ |
| 22 | —CH₂(CH₂)₂CH(CH₃)₂ |
| 23 | —CH(CH₃)CH(CH₃)CH₂CH₃ |
| 24 | —CH(CH₃)CH₂CH(CH₃)₂ |
| 25 | —CH₂CH(CH₃)CH(CH₃)₂ |
| 26 | —CH₂CH₂CH₂CH(CH₃)₂ |
| 27 | —C(CH₃)₂CH₂CH₂CH₃ |
| 28 | —C(CH₃)₂CH(CH₃)₂ |
| 29 | —C(CH₂CH₃)₂CH₃ |
| 30 | —CH₂CH(CH₂CH₃)₂ |
| 31 | 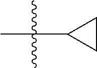 [SMILES: *C1CC1] |
| 32 | 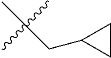 [SMILES: *CC1CC1] |
| 33 | 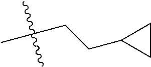 [SMILES: *CCC1CC1] |
| 34 | 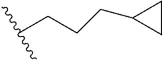 [SMILES: *CCCC1CC1] |
| 35 | 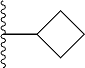 [SMILES: *C1CCC1] |
| 36 | 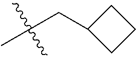 [SMILES: *CC1CCC1] |
| 37 | 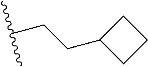 [SMILES: *CCC1CCC1] |
| 38 | 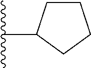 [SMILES: *C1CCCC1] |
| 39 | 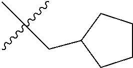 [SMILES: *CC1CCCC1] |
| 40 | 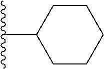 [SMILES: *C1CCCCC1] |
| 41 | 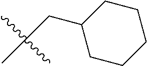 [SMILES: *CC1CCCCC1] |
| 42 | 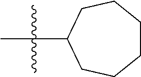 [SMILES: *C1CCCCCC1] |
| 43 | 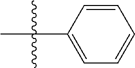 [SMILES: *c1ccccc1] |
| 44 | —CH₂CH*(CH₂CH═CHCH₂CH₂)** |
| 45 | —CH*(CH₂CH₂CH═CHCH₂CH₂)** |
| 47 | —CH₂CH*(CH═CHCH₂CH═CH)*** |
| 48 | —CH(CH₂CH₂CH₂NH)^{£} |
| 49 | —CH(CH═CHCH₂NH) ^{£} |
| 50 | —CH(CH₂CH═CHNH) ^{£} |
| 51 | —CH(CH₂CH₂CH═N) ^{£} |
| 52 | —C(═CHCH₂CH₂NH) ^{£} |
| 53 | —CH(CH₂CH₂CH₂O)^{£} |
| 54 | —CH(CH═CHCH₂O) ^{£} |
| 55 | —CH(CH₂CH═CHO) ^{£} |
| 56 | —C(═CHCH₂CH₂O) ^{£} |

In each of numbers 44-56 in Table A1, the parenthetical group forms a ring with the carbon immediately preceding the parenthetical. * The number of hydrogen on this carbon can be zero or 1 depending on the location of the double bond within the ring. ** The double bond can be at any position in the ring. *** The double bonds can be at any position in the ring and can be conjugated or non-conjugated. ^{£}The heteroatom can be at any position except bonded to the acyloxyl center carbon.

Table A2 provides illustrative examples in which the two of R⁴ are taken together with the atom(s) to which they are bound to form an optionally substituted 3-7 membered ring.

TABLE A2

| | |
|---|---|
| 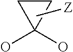 | Cyclopropyl |
| 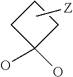 | Cyclobutyl |
| 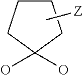 | Cyclopentyl |
| 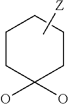 | Cyclohexyl |
| 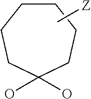 | Cycloheptyl |

wherein Z is H, alkyl, substituted alkyl, cycloalkyl, substituted cycloalkyl, aryl, substituted aryl, heteroaryl and substituted heteroaryl.

Table B1 below provides examples of R¹ where r and s are 0. Table B2 below provides examples of R¹ where r is 1 and s is 0. Table B3 below provides examples of R¹ where r and s are 1. In each of tables B1, B2 and B3, R′ is an amino acid side chain selected from any natural or non-standard amino acids.

TABLE B1

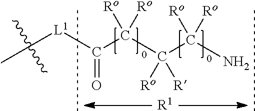

| # | R¹ where r and s are 0 |
|---|---|
| 1 | —C(O)C(R′)(H)NH₂ |
| 2 | —C(O)C(R′)(CH₃)NH₂ |
| 3 | —C(O)C(R′)(CH₂CH₃)NH₂ |
| 5 | —C(O)C(R′)(CH₂CH₂CH₃)NH₂ |
| 6 | —C(O)C(R′)(CH(CH₃)₂)NH₂ |
| 7 | —C(O)C(R′)(CH₂CH₂CH₂CH₃)NH₂ |
| 8 | —C(O)C(R′)(CH₂CH(CH₃)₂)NH₂ |
| 9 | —C(O)C(R′)(CH(CH₃)CH₂CH₃)NH₂ |
| 10 | —C(O)C(R′)(C(CH₃)₃)NH₂ |
| 11 | —C(O)C(R′)(CH₂(CH₂)₃CH₃)NH₂ |
| 12 | —C(O)C(R′)(CH(CH₃)(CH₂)₂CH₃)NH₂ |
| 13 | —C(O)C(R′)(CH(CH₃)CH(CH₃)₂)NH₂ |
| 14 | —C(O)C(R′)(C(CH₃)₂CH₂CH₃)NH₂ |
| 15 | —C(O)C(R′)(CH(CH₂CH₃)₂)NH₂ |
| 16 | —C(O)C(R′)(CH₂CH(CH₃)CH₂CH₃)NH₂ |
| 17 | —C(O)C(R′)(CH₂CH₂CH(CH₃)₂)NH₂ |
| 18 | —C(O)C(R′)(CH₂C(CH₃)₃)NH₂ |
| 19 | —C(O)C(R′)(—CH₂(CH₂)₄CH₃)NH₂ |
| 20 | —C(O)C(R′)(—CH(CH₃)(CH₂)₃CH₃)NH₂ |
| 21 | —C(O)C(R′)(—CH₂CH(CH₃)(CH₂)₂CH₃)NH₂ |
| 22 | —C(O)C(R′)(—CH₂CH₂CH(CH₃)CH₂CH₃)NH₂ |
| 23 | —C(O)C(R′)(—CH₂(CH₂)₂CH(CH₃)₂)NH₂ |
| 24 | —C(O)C(R′)(—CH(CH₃)CH(CH₃)CH₂CH₃)NH₂ |
| 25 | —C(O)C(R′)(—CH(CH₃)CH₂CH(CH₃)₂)NH₂ |
| 26 | —C(O)C(R′)(—CH₂CH(CH₃)CH(CH₃)₂)NH₂ |
| 27 | —C(O)C(R′)(—CH₂CH₂CH₂CH(CH₃)₂)NH₂ |
| 28 | —C(O)C(R′)(—C(CH₃)₂CH₂CH₂CH₃)NH₂ |
| 29 | —C(O)C(R′)(—C(CH₃)₂CH(CH₃)₂)NH₂ |
| 30 | —C(O)C(R′)(—C(CH₂CH₃)₂CH₃)NH₂ |
| 31 | —C(O)C(R′)(—CH₂CH(CH₂CH₃)₂)NH₂ |
| 32 | —C(O)C(R′)(—CH(CH₂)₂)NH₂ |
| 33 | —C(O)C(R′)(—CH₂CH(CH₂)₂)NH₂ |
| 34 | —C(O)C(R′)(—CH₂CH₂CH(CH₂)₂)NH₂ |
| 35 | —C(O)C(R′)(—CH₂CH₂CH₂CH(CH₂)₂)NH₂ |
| 36 | —C(O)C(R′)(—CH(CH₂)₃)NH₂ |
| 37 | —C(O)C(R′)(—CH₂CH(CH₂)₃)NH₂ |
| 38 | —C(O)C(R′)(—CH₂CH₂CH(CH₂)₃)NH₂ |
| 39 | —C(O)C(R′)(—CH(CH₂)₄)NH₂ |
| 40 | —C(O)C(R′)(—CH₂CH(CH₂)₄)NH₂ |
| 41 | —C(O)C(R′)(—CH(CH₂)₅)NH₂ |
| 42 | —C(O)C(R′)(—CH₂CH(CH₂)₅)NH₂ |
| 43 | —C(O)C(R′)(—CH(CH₂)₆)NH₂ |
| 44 | —C(O)C(R′)(—C₆H₅)NH₂ |

TABLE B2

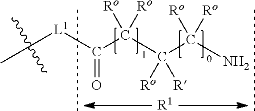

| # | R¹ where r is 1 and s is 0 |
|---|---|
| 45 | C(O)CH₂CH(R′)NH₂ |
| 46 | C(O)CH(CH₃)CH(R′)NH₂ |
| 47 | C(O)CH(CH₂CH₃)CH(R′)NH₂ |
| 48 | C(O)CH(CH₂CH₂CH₃)CH(R′)NH₂ |
| 49 | C(O)CH(CH₂CH₂CH₂CH₃)CH(R′)NH₂ |
| 50 | C(O)CH(CH₃)C(CH₃)(R′)NH₂ |
| 51 | C(O)CH(CH₃)C(CH₃CH₂)(R′)NH₂ |
| 52 | C(O)CH(CH₃)C(CH₃CH₂CH₂)(R′)NH₂ |
| 53 | C(O)CH(CH₃)C(CH₃CH₂CH₂CH₂)(R′)NH₂ |
| 54 | C(O)CH(CH₂CH₃)C(CH₃)(R′)NH₂ |
| 55 | C(O)CH(CH₂CH₃)C(CH₃CH₂)(R′)NH₂ |
| 56 | C(O)CH(CH₂CH₃)C(CH₃CH₂CH₂)(R′)NH₂ |
| 57 | C(O)CH(CH₂CH₃)C(CH₃CH₂CH₂CH₂)(R′)NH₂ |
| 58 | C(O)CH(CH₂CH₂CH₃)C(CH₃)(R′)NH₂ |
| 59 | C(O)CH(CH₂CH₂CH₃)C(CH₃CH₂)(R′)NH₂ |
| 60 | C(O)CH(CH₂CH₂CH₃)C(CH₃CH₂CH₂)(R′)NH₂ |
| 61 | C(O)CH(CH₂CH₂CH₃)C(CH₃CH₂CH₂CH₂)(R′)NH₂ |
| 62 | C(O)CH(CH₂CH₂CH₂CH₃)C(CH₃)(R′)NH₂ |
| 63 | C(O)CH(CH₂CH₂CH₂CH₃)C(CH₃CH₂)(R′)NH₂ |
| 64 | C(O)CH(CH₂CH₂CH₂CH₃)C(CH₃CH₂CH₂)(R′)NH₂ |
| 65 | C(O)CH(CH₂CH₂CH₂CH₃)C(CH₃CH₂CH₂CH₂)(R′)NH₂ |
| 66 | C(O)C(CH₃)(CH₂CH₂CH₂CH₃)CH(R′)NH₂ |
| 67 | C(O)C(CH₃)(CH₂CH₂CH₂CH₃)C(CH₃)(R′)NH₂ |
| 68 | C(O)C(CH₃)(CH₂CH₂CH₂CH₃)C(CH₃CH₂)(R′)NH₂ |
| 69 | C(O)C(CH₃)(CH₂CH₂CH₂CH₃)C(CH₃CH₂CH₂)(R′)NH₂ |
| 70 | C(O)C(CH₃)(CH₂CH₂CH₂CH₃)C(CH₃CH₂CH₂CH₂)(R′)NH₂ |
| 71 | C(O)C(CH₃CH₂)(CH₂CH₂CH₂CH₃)CH(R′)NH₂ |
| 72 | C(O)C(CH₃CH₂)(CH₂CH₂CH₂CH₃)C(CH₃)(R′)NH₂ |
| 73 | C(O)C(CH₃CH₂)(CH₂CH₂CH₂CH₃)C(CH₃CH₂)(R′)NH₂ |
| 74 | C(O)C(CH₃CH₂)(CH₂CH₂CH₂CH₃)C(CH₃CH₂CH₂)(R′)NH₂ |
| 75 | C(O)C(CH₃CH₂)(CH₂CH₂CH₂CH₃)C(CH₃CH₂CH₂CH₂)(R′)NH₂ |
| 76 | C(O)C(CH₃CH₂CH₂)(CH₂CH₂CH₂CH₃)CH(R′)NH₂ |
| 77 | C(O)C(CH₃CH₂CH₂)(CH₂CH₂CH₂CH₃)C(CH₃)(R′)NH₂ |
| 78 | C(O)C(CH₃CH₂CH₂)(CH₂CH₂CH₂CH₃)C(CH₃CH₂)(R′)NH₂ |
| 79 | C(O)C(CH₃CH₂CH₂)(CH₂CH₂CH₂CH₃)C(CH₃CH₂CH₂)(R′)NH₂ |
| 80 | C(O)C(CH₃CH₂CH₂)(CH₂CH₂CH₂CH₃)C(CH₃CH₂CH₂CH₂)(R′)NH₂ |
| 81 | C(O)C(CH₃CH₂CH₂CH₂)(CH₂CH₂CH₂CH₃)CH(R′)NH₂ |
| 82 | C(O)C(CH₃CH₂CH₂CH₂)(CH₂CH₂CH₂CH₃)C(CH₃)(R′)NH₂ |
| 83 | C(O)C(CH₃CH₂CH₂CH₂)(CH₂CH₂CH₂CH₃)C(CH₃CH₂)(R′)NH₂ |
| 84 | C(O)C(CH₃CH₂CH₂CH₂)(CH₂CH₂CH₂CH₃)C(CH₃CH₂CH₂)(R′)NH₂ |
| 85 | C(O)C(CH₃CH₂CH₂CH₂)(CH₂CH₂CH₂CH₃)C(CH₃CH₂CH₂CH₂)(R′)NH₂ |

TABLE B3

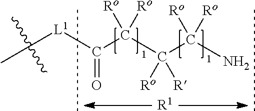

| # | R¹ where r is 1 and s is 1 |
|---|---|
| 86 | C(O)CH₂CH(R′)CH₂NH₂ |
| 87 | C(O)CH(CH₃)CH(R′)CH(CH₃)NH₂ |
| 88 | C(O)CH(CH₂CH₃)CH(R′)CH(CH₂CH₃)NH₂ |
| 89 | C(O)CH(CH₂CH₂CH₃)CH(R′)CH(CH₂CH₂CH₃)NH₂ |
| 90 | C(O)CH(CH₂CH₂CH₂CH₃)CH(R′)CH(CH₂CH₂CH₂CH₃)NH₂ |
| 91 | C(O)CH (CH₃)C(CH₃CH₂)(R′)CH(CH₃)(NH₂ |
| 92 | C(O)CH(CH₃)C(CH₃CH₂CH₂)(R′)CH(CH₃)(NH₂ |
| 93 | C(O)CH (CH₃)C(CH₃CH₂CH₂CH₂)(R′)CH(CH₃)(NH₂ |
| 94 | C(O)CH(CH₂CH₃)C(CH₃CH₂)(R′)CH(CH₂CH₃)NH₂ |
| 95 | C(O)CH(CH₂CH₃)C(CH₃CH₂CH₂)(R′)CH(CH₂CH₃)NH₂ |
| 96 | C(O)CH(CH₂CH₃)C(CH₃CH₂CH₂CH₂)(R′)CH(CH₂CH₃)NH₂ |
| 97 | C(O)CH(CH₂CH₂CH₃)C(CH₃)(R′)CH(CH₂CH₂CH₃)NH₂ |
| 98 | C(O)CH(CH₂CH₂CH₃)C(CH₃CH₂)(R′)CH(CH₂CH₂CH₃)NH₂ |
| 99 | C(O)CH(CH₂CH₂CH₃)C(CH₃CH₂CH₂)(R′)CH(CH₂CH₂CH₃)NH₂ |
| 100 | C(O)CH(CH₂CH₂CH₃)C(CH₃CH₂CH₂CH₂)(R′)CH(CH₂CH₂CH₃)NH₂ |
| 101 | C(O)CH(CH₂CH₂CH₂CH₃)C(CH₃)(R′)CH(CH₂CH₂CH₂CH₃)NH₂ |
| 102 | C(O)CH(CH₂CH₂CH₂CH₃)C(CH₃CH₂)(R′)CH(CH₂CH₂CH₂CH₃)NH₂ |
| 103 | C(O)CH(CH₂CH₂CH₂CH₃)C(CH₃CH₂CH₂)(R′)CH(CH₂CH₂CH₂CH₃)NH₂ |
| 104 | C(O)CH(CH₂CH₂CH₂CH₃)C(CH₃CH₂CH₂CH₂)(R′)CH(CH₂CH₂CH₂CH₃)NH₂ |
| 105 | C(O)C(CH₃)(CH₂CH₂CH₂CH₃)CH(R′)C(CH₃)(CH₂CH₂CH₂CH₃)NH₂ |
| 106 | C(O)C(CH₃)(CH₂CH₂CH₂CH₃)C(CH₃)(R′)C(CH₃)(CH₂CH₂CH₂CH₃)NH₂ |
| 107 | C(O)C(CH₃)(CH₂CH₂CH₂CH₃)C(CH₃CH₂)(R′)C(CH₃)(CH₂CH₂CH₂CH₃)NH₂ |
| 108 | C(O)C(CH₃)(CH₂CH₂CH₂CH₃)C(CH₃CH₂CH₂)(R′)C(CH₃)(CH₂CH₂CH₂CH₃)NH₂ |
| 109 | C(O)C(CH₃)(CH₂CH₂CH₂CH₃)C(CH₃CH₂CH₂CH₂)(R′)C(CH₃)(CH₂CH₂CH₂CH₃)—NH₂ |
| 110 | C(O)C(CH₃CH₂)(CH₂CH₂CH₂CH₃)CH(R′)C(CH₃CH₂)(CH₂CH₂CH₂CH₃)NH₂ |
| 111 | C(O)C(CH₃CH₂)(CH₂CH₂CH₂CH₃)C(CH₃)(R′)C(CH₃CH₂)(CH₂CH₂CH₂CH₃)NH₂ |
| 112 | C(O)C(CH₃CH₂)(CH₂CH₂CH₂CH₃)C(CH₃CH₂)(R′)C(CH₃CH₂)(CH₂CH₂CH₂CH₃)—NH₂ |
| 113 | C(O)C(CH₃CH₂)(CH₂CH₂CH₂CH₃)C(CH₃CH₂CH₂)(R′)C(CH₃CH₂)(CH₂CH₂CH₂—CH₃)NH₂ |
| 114 | C(O)C(CH₃CH₂)(CH₂CH₂CH₂CH₃)C(CH₃CH₂CH₂CH₂)(R′)C(CH₃CH₂)—(CH₂CH₂CH₂CH₃)NH₂ |
| 115 | C(O)C(CH₃CH₂CH₂)(CH₂CH₂CH₂CH₃)CH(R′)C(CH₃CH₂CH₂)(CH₂CH₂CH₂CH₃)—NH₂ |
| 116 | C(O)C(CH₃CH₂CH₂)(CH₂CH₂CH₂CH₃)C(CH₃)(R′)C(CH₃CH₂CH₂)(CH₂CH₂CH₂—CH₃)NH₂ |
| 117 | C(O)C(CH₃CH₂CH₂)(CH₂CH₂CH₂CH₃)C(CH₃CH2)(R′)C(CH₃CH₂CH₂)(CH₂CH₂—CH₂CH₃)NH₂ |
| 118 | C(O)C(CH₃CH₂CH₂)(CH₂CH₂CH₂CH₃)C(CH₃CH₂CH₂)(R′)C(CH₃CH₂CH₂)—(CH₂CH₂CH₂CH₃)NH₂ |
| 119 | C(O)C(CH₃CH₂CH₂)(CH₂CH₂CH₂CH₃)C(CH₃CH₂CH₂CH₂)(R′)C(CH₃CH₂CH₂)—(CH₂CH₂CH₂CH₃)NH₂ |
| 120 | C(O)C(CH₃CH₂CH₂CH₂)(CH₂CH₂CH₂CH₃)CH(R′)C(CH₃CH₂CH₂CH₂)(CH₂CH₂—CH₂CH₃)NH₂ |
| 121 | C(O)C(CH₃CH₂CH₂CH₂)(CH₂CH₂CH₂CH₃)C(CH₃)(R′)C(CH₃CH₂CH₂CH₂)(CH₂—CH₂CH₂CH₃)NH₂ |
| 122 | C(O)C(CH₃CH₂CH₂CH₂)(CH₂CH₂CH₂CH₃)C(CH₃CH₂)(R′)C(CH₃CH₂CH₂CH₂)—(CH₂CH₂CH₂CH₃)NH₂ |
| 123 | C(O)C(CH₃CH₂CH₂CH₂)(CH₂CH₂CH₂CH₃)C(CH₃CH₂CH₂)(R′)C(CH₃CH₂CH₂—CH₂)(CH₂CH₂CH₂CH₃)(NH₂ |
| 124 | C(O)C(CH₃CH₂CH₂CH₂)(CH₂CH₂CH₂CH₃)C(CH₃CH₂CH₂CH₂)(R′)C(CH₃CH₂—CH₂CH₂)(CH₂CH₂CH₂CH₃)NH₂ |
| 125 | C(O)CH(CH₃)CH(R′)CH(CH₃)NH₂ |
| 126 | C(O)CH(CH₂CH₃)CH(R′)CH(CH₂CH₃)NH₂ |
| 127 | C(O)CH(CH₂CH₂CH₃)CH(R′)CH(CH₂CH₂CH₃)NH₂ |
| 128 | C(O)CH(CH₂CH₂CH₂CH₃)CH(R′)CH(CH₂CH₂CH₂CH₃)NH₂ |
| 129 | C(O)CH(CH₃)C(CH₃)(R′)CH(CH₃)NH₂ |
| 130 | C(O)CH(CH₃)C(CH₃CH₂)(R′)CH(CH₃)NH₂ |
| 131 | C(O)CH(CH₃)C(CH₃CH₂CH₂)(R′)CH(CH₃)NH₂ |
| 132 | C(O)CH(CH₃)C(CH₃CH₂CH₂CH₂)(R′)CH(CH₃)NH₂ |
| 133 | C(O)CH(CH₂CH₃)C(CH₃)(R′)CH(CH₂CH₃)NH₂ |
| 134 | C(O)CH(CH₂CH₃)C(CH₃CH₂)(R′)CH(CH₂CH₃)NH₂ |
| 135 | C(O)CH(CH₂CH₃)C(CH₃CH₂CH₂)(R′)CH(CH₂CH₃)NH₂ |
| 136 | C(O)CH(CH₂CH₃)C(CH₃CH₂CH₂CH₂)(R′)CH(CH₂CH₃)NH₂ |
| 137 | C(O)CH(CH₂CH₂CH₃)C(CH₃)(R′)CH(CH₂CH₂CH₃)NH₂ |
| 138 | C(O)CH(CH₂CH₂CH₃)C(CH₃CH₂)(R′)CH(CH₂CH₂CH₃)NH₂ |
| 139 | C(O)CH(CH₂CH₂CH₃)C(CH₃CH₂CH₂)(R′)CH(CH₂CH₂CH₃)NH₂ |
| 140 | C(O)CH(CH₂CH₂CH₃)C(CH₃CH₂CH₂CH₂)(R′)CH(CH₂CH₂CH₃) NH₂ |
| 141 | C(O)CH(CH₂CH₂CH₂CH₃)C(CH₃)(R′)CH(CH₂CH₂CH₂CH₃)NH₂ |
| 142 | C(O)CH(CH₂CH₂CH₂CH₃)C(CH₃CH₂)(R′)CH(CH₂CH₂CH₂CH₃)NH₂ |
| 143 | C(O)CH(CH₂CH₂CH₂CH₃)C(CH₃CH₂CH₂)(R′)CH(CH₂CH₂CH₂CH₃)NH₂ |
| 144 | C(O)CH(CH₂CH₂CH₂CH₃)C(CH₃CH₂CH₂CH₂)(R′)CH(CH₂CH₂CH₂CH₃)NH₂ |
| 145 | C(O)C(CH₃)(CH₂CH₂CH₂CH₃)CH(R′)C(CH₃)(CH₂CH₂CH₂CH₃)NH₂ |
| 146 | C(O)C(CH₃)(CH₂CH₂CH₂CH₃)C(CH₃)(R′)C(CH₃)(CH₂CH₂CH₂CH₃)NH₂ |
| 147 | C(O)C(CH₃)(CH₂CH₂CH₂CH₃)C(CH₃CH₂)(R′)C(CH₃)(CH₂CH₂CH₂CH₃)NH₂ |
| 148 | C(O)C(CH₃)(CH₂CH₂CH₂CH₃)C(CH₃CH₂CH₂)(R′)C(CH₃)(CH₂CH₂CH₂CH₃)NH₂ |
| 149 | C(O)C(CH₃)(CH₂CH₂CH₂CH₃)C(CH₃CH₂CH₂CH₂)(R′)C(CH₃)(CH₂CH₂CH₂CH₃)—NH₂ |
| 150 | C(O)C(CH₃CH₂)(CH₂CH₂CH₂CH₃)CH(R′)C(CH₃CH₂)(CH₂CH₂CH₂CH₃)NH₂ |
| 151 | C(O)C(CH₃CH₂)(CH₂CH₂CH₂CH₃)C(CH₃)(R′)C(CH₃CH₂)(CH₂CH₂CH₂CH₃)NH₂ |
| 152 | C(O)C(CH₃CH₂)(CH₂CH₂CH₂CH₃)C(CH₃CH₂)(R′)C(CH₃CH₂)(CH₂CH₂CH₂CH₃)—NH₂ |
| 153 | C(O)C(CH₃CH₂)(CH₂CH₂CH₂CH₃)C(CH₃CH₂CH₂)(R′)C(CH₃CH₂)(CH₂CH₂CH₂—CH₃)NH₂ |
| 154 | C(O)C(CH₃CH₂)(CH₂CH₂CH₂CH₃)C(CH₃CH₂CH₂CH₂)(R′)C(CH₃CH₂)(CH₂CH₂—CH₂CH₃)NH₂ |
| 155 | C(O)C(CH₃CH₂CH₂)(CH₂CH₂CH₂CH₃)CH(R′)C(CH₃CH₂CH₂)(CH₂CH₂CH₂CH₃)—NH₂ |
| 156 | C(O)C(CH₃CH₂CH₂)(CH₂CH₂CH₂CH₃)C(CH₃)(R′)C(CH₃CH₂CH₂)(CH₂CH₂CH₂—CH₃)NH₂ |
| 157 | C(O)C(CH₃CH₂CH₂)(CH₂CH₂CH₂CH₃)C(CH₃CH₂)(R′)C(CH₃CH₂CH₂)(CH₂CH₂—CH₂CH₃)NH₂ |
| 158 | C(O)C(CH₃CH₂CH₂)(CH₂CH₂CH₂CH₃)C(CH₃CH₂CH₂)(R′)C(CH₃CH₂CH2)(CH₂—CH₂CH₂CH₃)NH₂ |
| 159 | C(O)C(CH₃CH₂CH₂)(CH₂CH₂CH₂CH₃)C(CH₃CH₂CH₂CH₂)(R′)C(CH₃CH₂CH₂)—(CH₂CH₂CH₂CH₃)NH₂ |
| 160 | C(O)C(CH₃CH₂CH₂CH₂)(CH₂CH₂CH₂CH₃)CH(R′)C(CH₃CH₂CH₂CH₂)(CH₂CH₂—CH₂CH₃)NH₂ |
| 161 | C(O)C(CH₃CH₂CH₂CH₂)(CH₂CH₂CH₂CH₃)C(CH₃)(R′)C(CH₃CH₂CH₂CH₂)(CH₂—CH₂CH₂CH₃)NH₂ |
| 162 | C(O)C(CH₃CH₂CH₂CH₂)(CH₂CH₂CH₂CH₃)C(CH₃CH₂)(R′)C(CH₃CH₂CH₂CH₂)—(CH₂CH₂CH₂CH₃)NH₂ |
| 163 | C(O)C(CH₃CH₂CH₂CH₂)(CH₂CH₂CH₂CH₃)C(CH₃CH₂CH₂)(R′)C(CH₃CH₂CH₂—CH₂)(CH₂CH₂CH₂CH₃)NH₂ |
| 164 | C(O)C(CH₃CH₂CH₂CH₂)(CH₂CH₂CH₂CH₃)C(CH₃CH₂CH₂CH₂)(R′)C(CH₃CH₂—CH₂CH₂)(CH₂CH₂CH₂CH₃)NH₂ |
| 165 | C(O)CH(CH₃)CH(R′)CH(CH₂CH₃)NH₂ |
| 166 | C(O)CH(CH₂CH₃)CH(R′)CH(CH₂CH₂CH₃)NH₂ |
| 167 | C(O)CH(CH₂CH₂CH₃)CH(R′)CH(CH₂CH₂CH₂CH₃)NH₂ |
| 168 | C(O)CH(CH₃)C(CH₃)(R′)CH(CH₂CH₃)NH₂ |
| 169 | C(O)CH(CH₃)C(CH₃CH₂)(R′)CH(CH₂CH₂CH₃)NH₂ |
| 170 | C(O)CH(CH₃)C(CH₃CH₂CH₂)(R′)CH(CH₂CH₂CH₂CH₃)NH₂ |
| 171 | C(O)CH(CH₃)C(CH₃CH₂CH₂CH₂)(R′)CH(CH₂CH₃)NH₂ |
| 172 | C(O)CH(CH₂CH₃)C(CH₃)(R′)CH(CH₂CH₂CH₃)NH₂ |
| 173 | C(O)CH(CH₂CH₃)C(CH₃CH₂)(R′)CH(CH₂CH₂CH₂CH₃)NH₂ |
| 174 | C(O)CH(CH₂CH₃)C(CH₃CH₂CH₂)(R′)CH(CH₃)NH₂ |
| 175 | C(O)CH(CH₂CH₃)C(CH₃CH₂CH₂CH₂)(R′)CH(CH₂CH₃)NH₂ |
| 176 | C(O)CH(CH₂CH₂CH₃)C(CH₃)(R′)CH(CH₂CH₂CH₃)NH₂ |
| 177 | C(O)CH(CH₂CH₂CH₃)C(CH₃CH₂)(R′)CH(CH₂CH₃)NH₂ |
| 178 | C(O)CH(CH₂CH₂CH₃)C(CH₃CH₂CH₂)(R′)CH(CH₃)NH₂ |
| 179 | C(O)CH(CH₂CH₂CH₃)C(CH₃CH₂CH₂CH₂)(R′)CH(CH₃)NH₂ |
| 180 | C(O)CH(CH₂CH₂CH₂CH₃)C(CH₃)(R′)CH(CH₂CH₃)NH₂ |
| 181 | C(O)CH(CH₂CH₂CH₂CH₃)C(CH₃CH₂)(R′)CH(CH₂CH₂CH₃)NH₂ |
| 182 | C(O)CH(CH₂CH₂CH₂CH₃)C(CH₃CH₂CH₂)(R′)CH(CH₂CH₂CH₂CH₃)NH₂ |
| 183 | C(O)CH(CH₂CH₂CH₂CH₃)C(CH₃CH₂CH₂CH₂)(R′)CH(CH₃)NH₂ |
| 184 | C(O)C(CH₃)(CH₂CH₂CH₂CH₃)CH(R′)C(CH₃)(CH₂CH₃)NH₂ |
| 185 | C(O)C(CH₃)(CH₂CH₂CH₂CH₃)C(CH₃)(R′)C(CH₃)(CH₃)NH₂ |
| 186 | C(O)C(CH₃)(CH₂CH₂CH₂CH₃)C(CH₃CH₂)(R′)C(CH₃)(CH₂CH₃)NH₂ |
| 187 | C(O)C(CH₃)(CH₂CH₂CH₂CH₃)C(CH₃CH₂CH₂)(R′)C(CH₃)(CH₂CH₂CH₃)NH₂ |
| 188 | C(O)C(CH₃)(CH₂CH₂CH₂CH₃)C(CH₃CH₂CH₂CH₂)(R′)C(CH₃)(CH₂CH₂CH₂CH₃)NH₂ |
| 189 | C(O)C(CH₃CH₂)(CH₂CH₂CH₂CH₃)CH(R′)C(CH₃CH₂)(CH₃)NH₂ |
| 190 | C(O)C(CH₃CH₂)(CH₂CH₂CH₂CH₃)C(CH₃)(R′)C(CH₃CH₂)(CH₂CH₃)NH₂ |
| 191 | C(O)C(CH₃CH₂)(CH₂CH₂CH₂CH₃)C(CH₃CH₂)(R′)C(CH₃CH₂)(CH₂CH₂CH₃)NH₂ |
| 192 | C(O)C(CH₃CH₂)(CH₂CH₂CH₂CH₃)C(CH₃CH₂CH₂)(R′)C(CH₃CH₂)(CH₂CH₂CH₂—CH₃)NH₂ |
| 193 | C(O)C(CH₃CH₂)(CH₂CH₂CH₂CH₃)C(CH₃CH₂CH₂CH₂)(R′)C(CH₃CH₂)(CH₃)NH₂ |
| 194 | C(O)C(CH₃CH₂CH₂)(CH₂CH₂CH₂CH₃)CH(R′)C(CH₃CH₂CH₂)(CH₂CH₃)NH₂ |
| 195 | C(O)C(CH₃CH₂CH₂)(CH₂CH₂CH₂CH₃)C(CH₃)(R′)C(CH₃CH₂CH₂)(CH₂CH₂CH₃)NH₂ |
| 196 | C(O)C(CH₃CH₂CH₂)(CH₂CH₂CH₂CH₃)C(CH₃CH2)(R′)C(CH₃CH₂CH₂)(CH₂CH₂—CH₂CH₃)NH₂ |
| 197 | C(O)C(CH₃CH₂CH₂)(CH₂CH₂CH₂CH₃)C(CH₃CH₂CH₂)(R′)C(CH₃CH₂CH₂)(CH₃)—NH₂ |
| 198 | C(O)C(CH₃CH₂CH₂)(CH₂CH₂CH₂CH₃)C(CH₃CH₂CH₂CH₂)(R′)C(CH₃CH₂CH₂)—(CH₂CH₃)NH₂ |
| 199 | C(O)C(CH₃CH₂CH₂CH₂)(CH₂CH₂CH₂CH₃)CH(R′)C(CH₃CH₂CH₂CH₂)(CH₂CH₂—CH₃)NH₂ |
| 200 | C(O)C(CH₃CH₂CH₂CH₂)(CH₂CH₂CH₂CH₃)C(CH₃)(R′)C(CH₃CH₂CH₂CH₂)(CH₃)—NH₂ |
| 201 | C(O)C(CH₃CH₂CH₂CH₂)(CH₂CH₂CH₂CH₃)C(CH₃CH₂)(R′)C(CH₃CH₂CH₂CH₂)—(CH₂CH₃)NH₂ |
| 202 | C(O)C(CH₃CH₂CH₂CH₂)(CH₂CH₂CH₂CH₃)C(CH₃CH₂CH₂)(R′)C(CH₃CH₂CH₂—CH₂)(CH₂CH₂CH₃)NH₂ |
| 203 | C(O)CH(CH₃)CH(R′)CH(CH₂CH₂CH₃)NH₂ |
| 204 | C(O)CH(CH₂CH₃)CH(R′)CH(CH₃)NH₂ |
| 205 | C(O)CH(CH₂CH₂CH₃)CH(R′)CH(CH₃)NH₂ |
| 206 | C(O)CH(CH₂CH₂CH₂CH₃)CH(R′)CH(CH₃) NH₂ |
| 207 | C(O)CH(CH₃)C(CH₃)(R′)CH(CH₂CH₂CH₃) NH₂ |
| 208 | C(O)CH(CH₃)C(CH₃CH₂)(R′)CH(CH₂CH₂CH₃)NH₂ |
| 209 | C(O)CH(CH₃)C(CH₃CH₂CH₂)(R′)CH(CH₂CH₂CH₃)NH₂ |
| 210 | C(O)CH(CH₃)C(CH₃CH₂CH₂CH₂)(R′)CH(CH₂CH₂CH₃)NH₂ |
| 211 | C(O)CH(CH₂CH₃)C(CH₃)(R′)CH(CH₂CH₂CH₃)NH₂ |
| 212 | C(O)CH(CH₂CH₃)C(CH₃CH₂)(R′)CH(CH₂CH₂CH₃)NH₂ |
| 213 | C(O)CH(CH₂CH₃)C(CH₃CH₂CH₂)(R′)CH(CH₂CH₂CH₃)NH₂ |
| 214 | C(O)CH(CH₂CH₃)C(CH₃CH₂CH₂CH₂)(R′)CH(CH₂CH₂CH₃)NH₂ |
| 215 | C(O)CH(CH₂CH₂CH₃)C(CH₃)(R′)CH(CH₂CH₂CH₂CH₃)NH₂ |
| 216 | C(O)CH(CH₂CH₂CH₃)C(CH₃CH₂)(R′)CH(CH₂CH₂CH₂CH₃)NH₂ |
| 217 | C(O)CH(CH₂CH₂CH₃)C(CH₃CH₂CH₂)(R′)CH(CH₂CH₂CH₂CH₃)NH₂ |
| 218 | C(O)CH(CH₂CH₂CH₃)C(CH₃CH₂CH₂CH₂)(R′)CH(CH₂CH₂CH₂CH₃)NH₂ |
| 219 | C(O)CH(CH₂CH₂CH₂CH₃)C(CH₃)(R′)CH(CH₂CH₂CH₃)NH₂ |
| 220 | C(O)CH(CH₂CH₂CH₂CH₃)C(CH₃CH₂)(R′)CH(CH₂CH₂CH₃)NH₂ |
| 221 | C(O)CH(CH₂CH₂CH₂CH₃)C(CH₃CH₂CH₂)(R′)CH(CH₂CH₂CH₃)NH₂ |
| 222 | C(O)CH(CH₂CH₂CH₂CH₃)C(CH₃CH₂CH₂CH₂)(R′)CH(CH₂CH₂CH₃)NH₂ |
| 223 | C(O)C(CH₃)(CH₂CH₂CH₂CH₃)CH(R′)C(CH₂CH₃)(CH₂CH₂CH₂CH₃)NH₂ |
| 224 | C(O)C(CH₃)(CH₂CH₂CH₂CH₃)C(CH₃)(R′)C(CH₂CH₃)(CH₂CH₂CH₂CH₃)NH₂ |
| 225 | C(O)C(CH₃)(CH₂CH₂CH₂CH₃)C(CH₃CH₂)(R′)C(CH₂CH₃)(CH₂CH₂CH₂CH₃)NH₂ |
| 226 | C(O)C(CH₃)(CH₂CH₂CH₂CH₃)C(CH₃CH₂CH₂)(R′)C(CH₂CH₃)(CH₂CH₂CH₂CH₃)—NH₂ |
| 227 | C(O)C(CH₃)(CH₂CH₂CH₂CH₃)C(CH₃CH₂CH₂CH₂)(R′)C(CH₂CH₃)(CH₂CH₂CH₂—CH₃)NH₂ |
| 228 | C(O)C(CH₃CH₂)(CH₂CH₂CH₂CH₃)CH(R′)C(CH₂CH₃CH₂)(CH₂CH₂CH₂CH₃)NH₂ |
| 229 | C(O)C(CH₃CH₂)(CH₂CH₂CH₂CH₃)C(CH₃)(R′)C(CH₂CH₃CH₂)(CH₂CH₂CH₂CH₃)—NH₂ |
| 230 | C(O)C(CH₃CH₂)(CH₂CH₂CH₂CH₃)C(CH₃CH₂)(R′)C(CH₃CH₂CH₂)(CH₂CH₂CH₂—CH₃)NH₂ |
| 231 | C(O)C(CH₃CH₂)(CH₂CH₂CH₂CH₃)C(CH₃CH₂CH₂)(R′)C(CH₃CH₂CH₂)(CH₂CH₂—CH₂CH₃)NH₂ |
| 232 | C(O)C(CH₃CH₂)(CH₂CH₂CH₂CH₃)C(CH₃CH₂CH₂CH₂)(R′)C(CH₃CH₂CH₂)(CH₂CH₂CH₂CH₃)NH₂ |
| 233 | C(O)C(CH₃CH₂CH₂)(CH₂CH₂CH₂CH₃)CH(R′)(CH₃CH₂CH2cH₂)C(CH₂CH₂CH₂—CH₃)NH₂ |
| 234 | C(O)C(CH₃CH₂CH₂)(CH₂CH₂CH₂CH₃)C(CH₃)(R′)C(CH₃CH₂CH2cH₂)(CH₂CH₂CH₂—CH₃)NH₂ |
| 235 | C(O)C(CH₃CH₂CH₂)(CH₂CH₂CH₂CH₃)C(CH₃CH₂)(R′)C(CH₃CH₂CH₂CH₂)(CH₂CH₂—CH₂CH₃)NH₂ |
| 236 | C(O)C(CH₃CH₂CH₂)(CH₂CH₂CH₂CH₃)C(CH₃CH₂CH₂)(R′)C(CH₃CH₂CH₂CH₂)(CH₂—CH₂CH₂CH₃)NH₂ |
| 237 | C(O)C(CH₃CH₂CH₂)(CH₂CH₂CH₂CH₃)C(CH₃CH₂CH₂CH₂)(R′)C(CH₃CH₂CH₂—CH₂)—(CH₂CH₂CH₂CH₃)NH₂ |
| 238 | C(O)C(CH₃CH₂CH₂CH₂)(CH₂CH₂CH₂CH₃)CH(R′)C(CH₃CH₂CH₂CH₂)(CH₂CH₂—CH₂CH₃)NH₂ |
| 239 | C(O)C(CH₃CH₂CH₂CH₂)(CH₂CH₂CH₂CH₃)C(CH₃)(R′)C(CH₃CH₂CH₂CH₂)(CH₂—CH₂CH₂CH₃)NH₂ |
| 240 | C(O)C(CH₃CH₂CH₂CH₂)(CH₂CH₂CH₂CH₃)C(CH₃CH₂)(R′)C(CH₃CH₂CH₂CH₂)—(CH₂CH₂CH₂CH₃)NH₂ |
| 241 | C(O)C(CH₃CH₂CH₂CH₂)(CH₂CH₂CH₂CH₃)C(CH₃CH₂CH₂)(R′)C(CH₃CH₂CH₂)—(CH₂CH₂CH₂CH₃)NH₂ |
| 242 | C(O)C(CH₃CH₂CH₂CH₂)(CH₂CH₂CH₂CH₃)C(CH₃CH₂CH₂CH₂)(R′)C(CH₃CH₂—CH₂)—(CH₂CH₂CH₂CH₃)NH₂ |
| 243 | C(O)CH(CH₃)CH(R′)CH(CH₃)NH₂ |
| 244 | C(O)CH(CH₂CH₃)CH(R′)CH(CH₂CH₃)NH₂ |
| 245 | C(O)CH(CH₂CH₂CH₃)CH(R′)CH(CH₂CH₂CH₃)NH₂ |
| 246 | C(O)CH(CH₂CH₂CH₂CH₃)CH(R′)CH(CH₂CH₂CH₂CH₃)NH₂ |
| 247 | C(O)CH(CH₃)C(CH₃)(R′)CH(CH₃)NH₂ |
| 248 | C(O)CH(CH₃)(CH₃CH₂)C(R′)CH(CH₃)NH₂ |
| 249 | C(O)CH(CH₃)C(CH₃CH₂CH₂)(R′)CH(CH₃)NH₂ |
| 250 | C(O)CH(CH₃)C(CH₃CH₂CH₂CH₂)(R′)CH(CH₃)NH₂ |
| 251 | C(O)CH(CH₂CH₃)C(CH₃)(R′)CH(CH₂CH₃)NH₂ |
| 252 | C(O)CH(CH₂CH₃)C(CH₃CH₂)(R′)CH(CH₂CH₃)NH₂ |
| 253 | C(O)CH(CH₂CH₃)C(CH₃CH₂CH₂)(R′)CH(CH₂CH₃)NH₂ |
| 254 | C(O)CH(CH₂CH₃)C(CH₃CH₂CH₂CH₂)(R′)CH(CH₂CH₃)NH₂ |
| 255 | C(O)CH(CH₂CH₂CH₃)C(CH₃)(R′)CH(CH₂CH₂CH₃)NH₂ |
| 256 | C(O)CH(CH₂CH₂CH₃)C(CH₃CH₂)(R′)CH(CH₂CH₂CH₃)NH₂ |
| 257 | C(O)CH(CH₂CH₂CH₃)C(CH₃CH₂CH₂)(R′)CH(CH₂CH₂CH₃)NH₂ |
| 258 | C(O)CH(CH₂CH₂CH₃)C(CH₃CH₂CH₂CH₂)(R′)CH(CH₂CH₂CH₃)NH₂ |
| 259 | C(O)CH(CH₂CH₂CH₂CH₃)C(CH₃)(R′)CH(CH₂CH₂CH₂CH3NH₂) |
| 260 | C(O)CH(CH₂CH₂CH₂CH₃)C(CH₃CH₂)(R′)CH(CH₂CH₂CH₂CH₃)NH₂ |
| 261 | C(O)CH(CH₂CH₂CH₂CH₃)C(CH₃CH₂CH₂)(R′)CH(CH₂CH₂CH₂CH₃)NH₂ |
| 262 | C(O)CH(CH₂CH₂CH₂CH₃)C(CH₃CH₂CH₂CH₂)(R′)CH(CH₂CH₂CH₂CH₃)NH₂ |
| 263 | C(O)C(CH₃)(CH₂CH₂CH₂CH₃)CH(R′)C(CH₃)(CH₂CH₂CH₂CH₃)NH₂ |
| 264 | C(O)C(CH₃)(CH₂CH₂CH₂CH₃)C(CH₃)(R′)C(CH₃)(CH₂CH₂CH₂CH₃)NH₂ |
| 265 | C(O)C(CH₃)(CH₂CH₂CH₂CH₃)C(CH₃CH₂)(R′)C(CH₃)(CH₂CH₂CH₂CH₃)NH₂ |
| 266 | C(O)C(CH₃)(CH₂CH₂CH₂CH₃)C(CH₃CH₂CH₂)(R′)C(CH₃)(CH₂CH₂CH₂CH₃)NH₂ |
| 267 | C(O)C(CH₃)(CH₂CH₂CH₂CH₃)C(CH₃CH₂CH₂CH₂)(R′)C(CH₃)(CH₂CH₂CH₂CH₃)—NH₂ |
| 268 | C(O)C(CH₃CH₂)(CH₂CH₂CH₂CH₃)CH(R′)C(CH₃CH₂)(CH₂CH₂CH₂CH₃)NH₂ |
| 269 | C(O)C(CH₃CH₂)(CH₂CH₂CH₂CH₃)C(CH₃)(R′)C(CH₃CH₂)(CH₂CH₂CH₂CH₃)NH₂ |
| 270 | C(O)C(CH₃CH₂)(CH₂CH₂CH₂CH₃)C(CH₃CH₂)(R′)C(CH₃CH₂)(CH₂CH₂CH₂CH₃)—NH₂ |
| 271 | C(O)C(CH₃CH₂)(CH₂CH₂CH₂CH₃)C(CH₃CH₂CH₂)(R′)C(CH₃CH₂)(CH₂CH₂CH₂CH₃) NH₂ |
| 272 | C(O)C(CH₃CH₂)(CH₂CH₂CH₂CH₃)C(CH₃CH₂CH₂CH₂)(R′)C(CH₃CH₂) (CH₂CH₂CH₂CH₃) NH₂ |
| 273 | C(O)C(CH₃CH₂CH₂)(CH₂CH₂CH₂CH₃)CH(R′)C(CH₃CH₂CH₂)(CH₂CH₂CH₂CH₃)NH₂ |
| 274 | C(O)C(CH₃CH₂CH₂)(CH₂CH₂CH₂CH₃)C(CH₃)(R′)C(CH₃CH₂CH₂)(CH₂CH₂CH₂CH₃) NH₂ |
| 275 | C(O)C(CH₃CH₂CH₂)(CH₂CH₂CH₂CH₃)C(CH₃CH₂)(R′)C(CH₃CH₂CH₂) (CH₂CH₂CH₂CH₃) NH₂ |
| 276 | C(O)C(CH₃CH₂CH₂)(CH₂CH₂CH₂CH₃)C(CH₃CH₂CH₂)(R′)C(CH₃CH₂CH₂)(CH₂CH₂CH₂CH₃) NH₂ |
| 277 | C(O)C(CH₃CH₂CH₂)(CH₂CH₂CH₂CH₃)C(CH₃CH₂CH₂CH₂)(R′)C(CH₃CH₂CH₂)(CH₂CH₂CH₂CH₃) NH₂ |
| 278 | C(O)C(CH₃CH₂CH₂CH₂)(CH₂CH₂CH₂CH₃)CH(R′)C(CH₃CH₂CH₂CH₂)(CH₂CH₂CH₂CH₃) NH₂ |
| 279 | C(O)C(CH₃CH₂CH₂CH₂)(CH₂CH₂CH₂CH₃)C(CH₃)(R′)C(CH₃CH₂CH₂CH₂)(CH₂CH₂CH₂CH₃) NH₂ |
| 280 | C(O)C(CH₃CH₂CH₂CH₂)(CH₂CH₂CH₂CH₃)C(CH₃CH₂)(R′)C(CH₃CH₂CH₂CH₂)(CH₂CH₂CH₂CH₃) NH₂ |
| 281 | C(O)C(CH₃CH₂CH₂CH₂)(CH₂CH₂CH₂CH₃)C(CH₃CH₂CH₂)(R′)C(CH₃CH₂CH₂—CH₂) (CH₃CH₂CH₂CH₂)NH₂ |
| 282 | C(O)C(CH₃CH₂CH₂CH₂)(CH₂CH₂CH₂CH₃)C(CH₃CH₂CH₂CH₂)(R′)C(CH₃CH₂—CH₂CH₂)(CH₂CH₂CH₂CH₃) NH₂ |

All combinations of L¹, R⁴, R¹, R^{o}, R′, R″ and R′″ groups are contemplated in the present invention. The provided examples are not meant to be limiting but rather are provided to better illustrate the invention.

In certain embodiment of the invention R¹ is —C(O)(CR^{o}R^{o})ᵣC(R^{o}R′)(CR^{o}R^{o})ₛNH₂. In another embodiment R¹ is —C(O)(CR^{o}R^{o})ᵣC(R^{o}R′)(CR^{o}R^{o})ₛN(H)C(O)(CR^{o}R^{o})_{w}C(R^{o}R″)(CR^{o}R^{o})ₓNH₂. In yet another embodiment R¹ is —C(O)CH(R′)N(H)C(O)CH(R″)N(H)C(O)C(R^{o}R″′)NH₂.

In particular embodiments R¹ is —C(O)C(R^{o}R′)NH₂ and R⁴ is C₂-C₆ alkyl group. In other embodiments m, n and o are zero, R⁴ is —CH(CH₃)₂ and R¹ is —C(O)CH(CH₃)₂ and R′ is —CH₃, —CH(CH₃)₂, —CH₂CH(CH₃)₂, or —CH₂CH(CH₂CH₃)(CH₃). In one embodiment R³ is —CR*R**, R² is —NHC(NH₂)NH and m, n and o are zero.

In one embodiment R¹ is —C(O)CH(R)NH₂, R′ is —CH(CH₃)₂, R² is —NHC(NH₂)NH, R³ is —CH(OH)CH₂(OH)CH₂(OH) and R⁴ is —CH(CH₃). In another embodiment R¹ is —C(O)CH(R)NH₂, R′ is —CH(CH₃)₂, R² is —NHC(NH₂)NH, R³ is —CH(OCH₃)CH₂(OH)CH₂(OH) and R⁴ is —CH(CH₃)₂. In another embodiment R¹ is —C(O)CH(R′)NH₂, R′ is —CH(CH₃)₂, R² is —NHC(NH₂)NH, R³ is —CH(OCH₃)CH₂(OH)CH₂OC(O)(CH₂)₆CH₃ and R⁴ is —CH(CH₃)₂.

In some embodiments L¹ is —(CH₂)ₘC(R⁴)₂(CH₂)ₙO(CH₂)ₒ— and R′ is —CH(CH₃)₂ or —CH₂CH(CH₃)₂. In other embodiments L¹ is —C(R⁴)₂O— and R′ is —CH₃, —CH(CH₃)₂, —CH₂CH(CH₃)₂, or —CH₂CH(CH₂CH₃)(CH₃).

In certain embodiments of formula (III), R³ is —CH(OH)CH(OH)CH₂(OH) and R² is —NHC(NH₂)NH. In another embodiment R³ is —CH(OCH₃)CH(OH)CH₂(OH) and R² is —NHC(NH₂)NH. In yet another embodiment R³ is —C(OCH₃)C(OH)CHOC(O)(CH₂)₆CH₃ and R² is —NHC(NH₂)NH.

In any of the above recited embodiments R¹ is —C(O)CH(R′)NH₂ and R′ is —CH(CH₃)₂, or R¹ is —C(O)CH(R′)NH₂, and R′ is —CH(CH₃)₂, R² is —NHC(NH₂)NH, R³ is —CH(OCH₃)CH₂(OH)CH₂(OH) and R⁴ is —CH(CH₃)₂. In another embodiment R¹ is —C(O)CH(R′)NH₂, R′ is —CH(CH₃)₂, R² is —NHC(NH₂)NH, R³ is —CH(OCH₃)CH₂(OH)CH₂OC(O)(CH₂)₆CH₃ and R⁴ is —CH(CH₃)₂.

The compounds of the present disclosure can be formulated as pharmaceutical compositions and administered to a mammalian subject, such as a human patient in a variety of forms adapted to the chosen route of administration, i.e., orally, parenterally, by intravenous, intramuscular, topical or subcutaneous routes.

Thus, the present compounds may be systemically administered, e.g., orally, in combination with a pharmaceutically acceptable vehicle such as an inert diluent or an assimilable edible carrier.

Suitable dosage forms for oral administration include, for example, solid, semi-solid and liquid systems such as in hard or soft shell gelatin capsules, tablets, liquids, powders, lozenges (including liquid-filled), chews, gels, films, ovules, sprays, elixirs, suspensions, syrups, buccal/mucoadhesive patches and the like.

Oral dosage forms may, for example, contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices. The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form must be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

For topical administration, the present compounds may be applied in pure form, i.e., when they are liquids. However, it will generally be desirable to administer them to the skin as compositions or formulations, in combination with a dermatologically acceptable carrier, which may be a solid or liquid. Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user. Examples of useful dermatological compositions which can be used to deliver the compounds of the present disclosure to the skin are known to the art; for example, see Jacquet et al. U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compounds of the present disclosure can be determined by comparing their in vitro activity, and in vivo activity in animal models. Methods for the extrapolation of effective dosages in mice, and other animals, to humans are known to the art; for example, see U.S. Pat. No. 4,938,949.

The amount of the compound, or an active salt or derivative thereof, required for use in treatment will vary, for example, with the route of administration, the nature of the condition being treated and the age and condition of the patient and will be ultimately at the discretion of the attendant physician or clinician. In general, however, a suitable dose will be in the range of from about 0.01 to about 200 mg/kg, e.g., from about 0.01 to about 75 mg/kg of body weight per day, such as 0.01 to about 50 mg per kilogram body weight of the recipient per day, preferably in the range of 0.01 to 25 mg/kg/day, most preferably in the range of 0.01 to 10 mg/kg/day. The compound may conveniently be administered in unit dosage form; for example, containing from about 1 to about 2000 mg, conveniently about 1 to about 1000 mg, or about 1 to about 750 mg of active ingredient per unit dosage form. The desired dose may conveniently be presented in a single dose or as divided doses administered at appropriate intervals, for example, as two, three, four, or more sub-doses per day.

Embodiments of compounds, compositions and methods are illustrated in the following examples. These examples are provided for illustrative purposes and are not considered limitations on the scope of compounds, compositions and methods of the present disclosure.

### EXAMPLES

Compounds of formula (II) may be prepared by a number of synthetic routes. One such route is outlined in the following scheme:

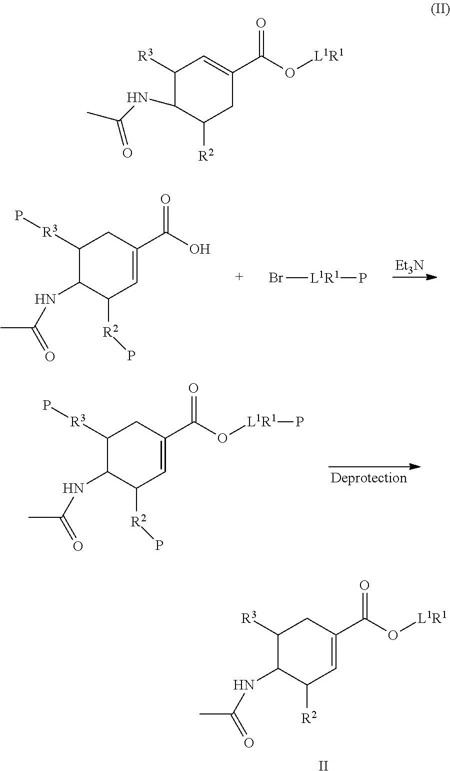

wherein P is a protecting group;
and L′, R¹, R², and R³ are as defined for formula (I).

### Example 1

### Preparation of an Isopropyl-Valine Analog of 4-Guanidinio Oseltamivir Carboxylate (GOC-Isp-Val)

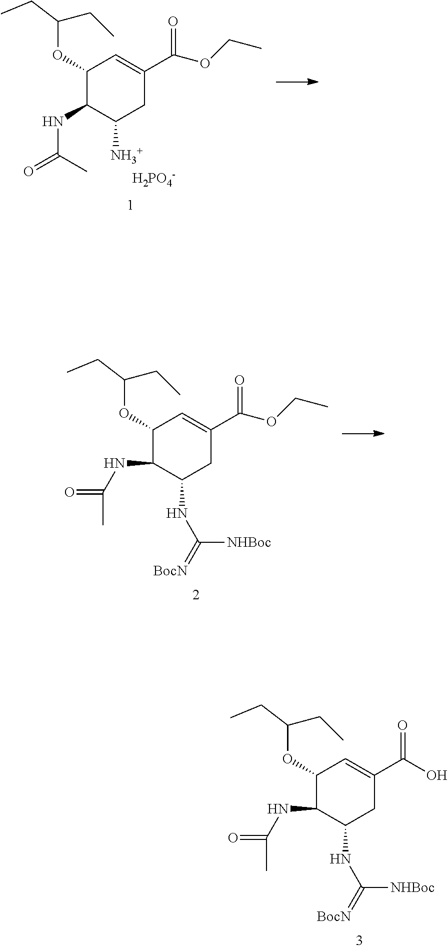

Compound 2:

1.13 g (3.65 mmole) N,N′-bis-Boc-1-Guanylpyrazole was added to a suspension of 1.5 g (3.65 mmole) oseltamivir monophosphate (1) in 20 ml anhydrous acetonitrile. After addition of 1.2 ml (8.7 mmole) of triethyl amine, the suspension was stirred at room temperature for 18 hours. All volatile components were removed by vacuum. The residue was purified by 100 g silica gel flash chromatography. 2 g of purified compound (2) was obtained with yield of 98%.

1H NMR (DMSO-d6) δ 0.787-0.864 (6H, m), 1.216-1.251 (3H, t), 1.355-1.396 (22H, m), 1.799 (3H, s), 2.318-2.344 (1H, m), 2.659-2.672 (1H, m), 3.400-3.428 (1H, m), 3.959-4.057 (2H, m), 4.134-4.232 (3H, m), 6.661 (1H, s), 7.893-7.913 (1H, d), 8.541-8.561 (1H, d), 11.523 (1H, s).

Mass spectrum: calculated for C₂₇H₄₆N₄O₈: 554.68. MS: m/z 550.20 (M+1).

Compound 3:

8.5 ml 1.46M KOH aqueous solution was added to a solution of 1.74 g (3.1 mmole) compound (2) in 12 ml tetrahydrofuran and 4 ml methanol. The mixture was stirred at room temperature overnight. All volatile components were removed by vacuum. 200 ml 0.1M phosphate buffer at pH of 6 was added to the white solid and, after stirring for 10 minutes, 0.1M potassium bisulfate was added dropwise carefully to adjust the pH to around 4.5 at which point a white precipitate was formed. 200 ml dichloromethane was added to dissolve all precipitate. The mixture was transferred to a separatory funnel and the dichloromethane layer was separated and washed with 100 ml water and 100 ml brine. The organic layer was dried over anhydrous sodium sulfate and the dichloromethane solvent was removed by vacuum. 1.32 g compound (3) was obtained with 80% yield.

1H NMR (DMSO-d6) δ 0.787-0.862 (6H, m), 1.397-1.494 (22H, m), 1.800 (3H, s), 2.234-2.295 (1H, m), 3.389-3.417 (1H, m), 3.946-4.057 (2H, m), 4.170-4.202 (1H, m), 6.714 (1H, s), 7.882-7.902 (1H, d), 8.530-8.549 (1H, d), 11.446 (1H, s), 12.700 (1H, br).

Mass spectrum: calculated for C₂₅H₄₂N₄O₈: 526.62. MS: m/z 527.20 (M+1).

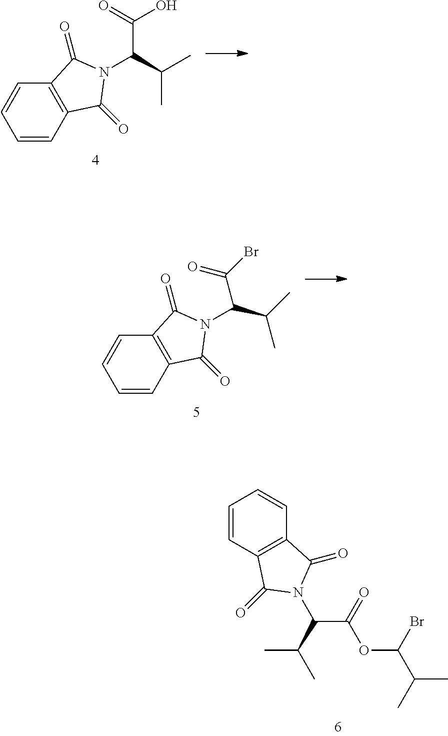

Compound 6:

13 ml of 1M oxalyl bromide in dichloromethane (DCM) was added to a solution of 5 g phthaloyl valine (4) in 20 ml anhydrous DCM. After 78 uL of anhydrous dimethylformamide was added, the reaction was stirred overnight until bubbling ceased. The volatile components were removed by evaporation under argon. The residue (5) was re-dissolved in 10 ml anhydrous DCM and mixed with a catalytical amount of anhydrous zinc chloride. After the temperature was lowered to −10° C. with ice-salt-water bath, 1.45 g isobutyraldehyde was added dropwise in 30 minutes. The reaction mixture was stirred at −5 to 5° C. for another 4 hours. The volatile components were removed by vacuum evaporation. The residue was subjected to a 100 g silica gel flash chromatography with 3:2 Hexane and ethyl acetate as eluent. 2.3 g of purified compound (6) was obtained with a yield of 30%.

1H NMR (DMSO-d6) δ 0.881-1.278 (12H, m), 2.004-2.104 (1H, m), 2.765-2.835 (1H, m), 4.602-4.652 (1H, m), 6.525-6.580 (1H, 2d), 7.733-7.807 (2H, m), 7.857-7.935 (2H, m).

Mass spectrum: calculated for C₁₇H₂₀BrNO₄: 382.25. MS: m/z 405.28 (M+Na⁺).

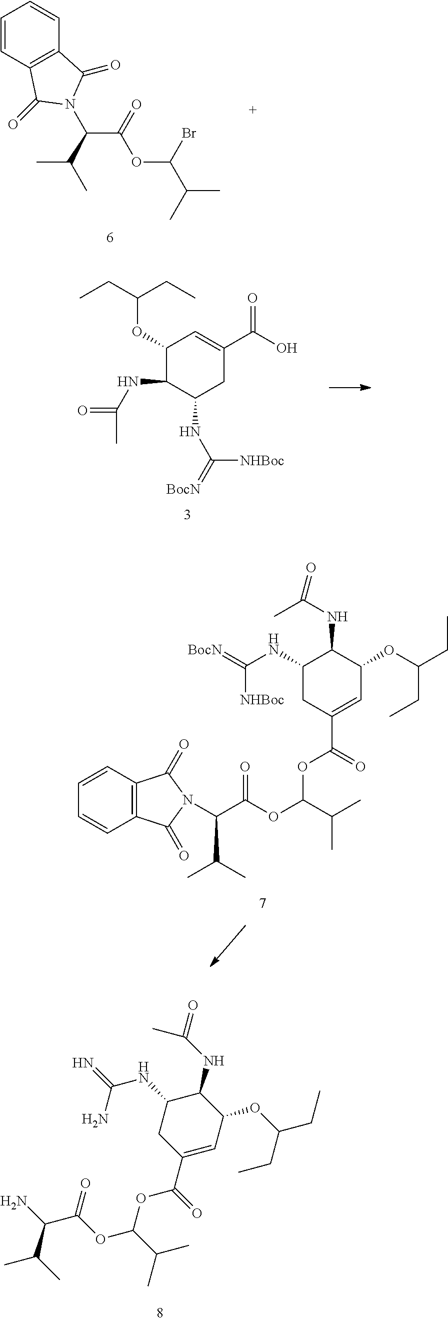

Compound 7:

0.3 ml triethylamine was added in one portion to solution of 0.5 g of compound (3) and 1.2 g compound (6) in 20 ml anhydrous acetonitrile. The solution was heated at 80° C., stirred and refluxed for 3 hours before all of the volatile components were removed by vacuum evaporation. The residue was subjected to a 100 g silica gel flash chromatography with 1:2 Hexane and ethyl acetate as eluent. 300 mg of purified compound 7 was obtained with yield of 40%.

1H NMR (DMSO-d6) δ 0.837-0.903 (18H, m), 1.495-1.508 (22H, m), 1.921-1.929 (3H, m), 2.250-2.320 (1H, m), 2.650-2.766 (2H, m), 3.368-3.395 (1H, m), 4.095-4.149 (2H, m), 4.310-4.395 (1H, m), 4.582-4.603 (1H, m), 5.302 (1H, s), 6.211-6.273 (1H, m), 6.684-6.743 (1H, m), 6.829-6.853 (1H, m), 7.759-7.793 (2H, m), 7.859-7.890 (2H, m), 8.580-8.621 (1H, m), 11.401 (1H, s).

Mass spectrum: calculated for C₄₂H₆₁N₅O₁₂: 827.96. MS: m/z 828.30 (M+1).

Compound 8:

4.67 ml of 0.3M hydrazine monohydrate in absolute ethanol was added to a solution of 250 mg of compound (7) in 2 ml absolute ethanol. After stirring at room temperature for one hour, 5 ml trifluoroacetic acid was added and the solution was stirred for another 4 hours. After removal of all volatile components by vacuum evaporation, the residue was subjected to a 100 g reverse phase silica gel flash column with dichloromethane and methanol (9:1 to 8:2). The collected sample was then further purified by reverse phase preparative HPLC with eluent of acetonitrile in water in a gradient of 0% to 90% plus 0.02% TFA in 30 minutes. 87 mg of purified compound (8) was obtained with yield of 59%.

1H NMR (D₂O) δ 0.842-0.910 (18H, m), 1.500 (4H, m), 1.949-1.960 (3H, m), 2.239-2.307 (1H, m), 2.684-2.792 (2H, m), 3.385-4.401 (1H, m), 4.121-4.172 (2H, m), 4.305-4.373 (1H, m), 4.602-4.616 (1H, m), 5.305 (1H, s), 6.664-6.793 (1H, m), 6.857-6.889 (1H, br), 8.600-8.628 (1H, m).

Mass spectrum: calculated for C₂₄H₄₃N₅O₆: 497.63. MS: m/z 498.27 (M+1).

Analytical Calculation for C₂₄H₄₃N₅O₆.3TFA: C, 42.92; H, 5.52; N, 8.34. Found by elemental analysis: C, 43.22; H, 5.58; N, 8.27.

### Example 2

### Preparation of a Methyl-Valine Analog of 4-Guanidinio Oseltamivir Carboxylate (GOC-Me-Valine)

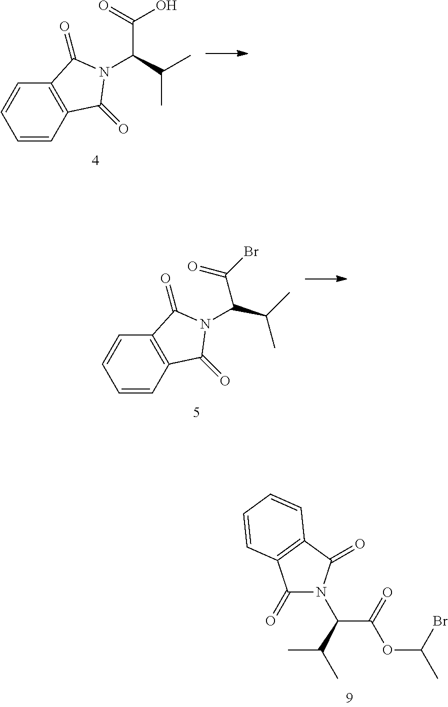

Compound 9:

13 ml 1M oxalyl bromide in Dichloromethane (DCM) was added to a solution of 5 g phthaloyl valine (4) in 20 ml anhydrous DCM. After 78 uL of anhydrous dimethylformamide was added, the reaction was stirred overnight until bubbling ceased. The volatile components were removed by evaporation under argon. The residue (5) was re-dissolved in 10 ml anhydrous DCM and mixed with a catalytic amount of anhydrous zinc chloride. After the temperature was lowered to −10° C. with ice-salt-water bath, 1.16 g acetaldehyde was added dropwise in 30 minutes. The reaction mixture was stirred at −5 to 5° C. for another 4 hours. The volatile components were removed by vacuum evaporation. The residue was subjected to a 100 g silica gel flash chromatography with 3:2 Hexane and ethyl acetate as eluent. 5.68 g of purified compound (9) was obtained with yield of 79%.

1H NMR (DMSO-d6) δ 0.909-0.943 (3H, t), 1.153-1.170 (3H, d), 1.856-1.870 (3H, d), 2.783-2.852 (1H, m), 4.580-4.639 (1H, m), 6.690-6.794 (1H, m), 7.774-7.794 (2H, m), 7.865-7.918 (2H, m).

Mass spectrum: calculated for C₁₅H₁₆BrNO₄: 354.20. MS: m/z 377.20 (M+Na⁺).

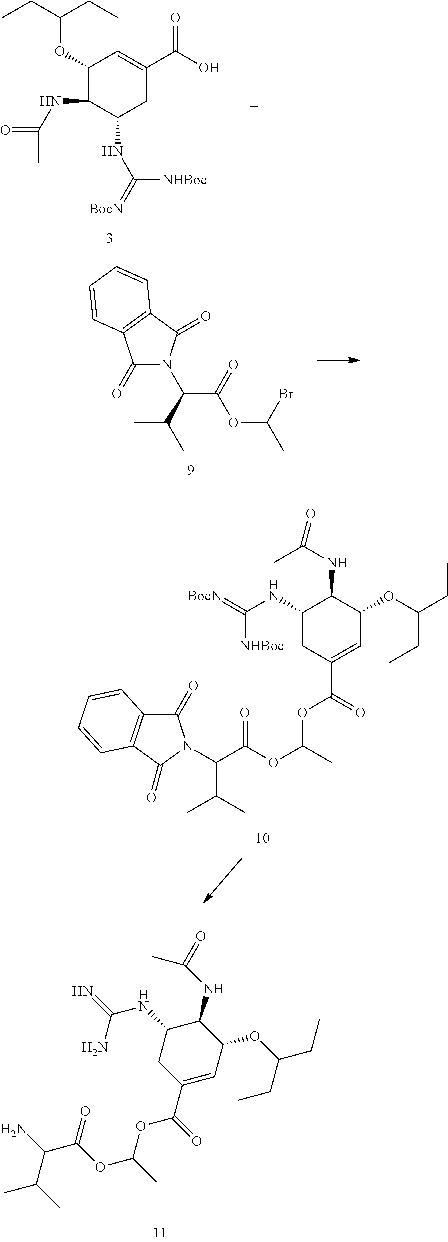

Compound 10:

0.3 ml triethylamine was added in one portion to solution of 0.5 g of compound (3) and compound 1.0 g compound (9) in 20 ml anhydrous acetonitrile. The solution was heated at 80° C., stirred and refluxed for 3 hours before all of the volatile components were removed by vacuum evaporation. The residue was subjected to a 100 g silica gel flash chromatography with 1:2 Hexane and ethyl acetate as eluent. 550 mg of purified compound 10 was obtained with a yield of 70%.

1H NMR (D₂O) δ 0.861-1.173 (15H, m), 1.467-1.522 (22H, m), 1.916-1.935 (3H, m), 2.180-2.398 (1H, m), 2.619-2.994 (2H, m), 3.320-3.451 (1H, m), 3.980-4.149 (2H, m), 4.336-4.460 (1H, m), 4.511-4.604 (1H, m), 6.160-6.274 (1H, m), 6.696-6.884 (1H, m), 6.898-6.990 (1H, m), 7.716-7.789 (2H, m), 7.815-7.893 (2H, m), 8.537-8.638 (1H, m), 11.397-11.406 (1H, m).

Mass spectrum: calculated for C₄₀H₅₇N₅O₁₂: 799.91. MS: m/z 801.10 (M+1).

Compound 11:

4.67 ml of 0.3M hydrazine monohydrate in absolute ethanol was added to a solution of 450 mg of compound (10) in 2 ml absolute ethanol. After stirring at room temperature for one hour, 5 ml trifluoroacetic acid was added and the solution was stirred for another 4 hours. After removal of all volatile components by vacuum evaporation, the residue was subjected to a 100 g reverse phase silica gel flash column chromatography with dichloromethane and methanol (9:1 to 8:2). The collected sample was then further purified by reverse phase preparative HPLC with eluent of acetonitrile in water in a gradient method of 0% to 90% plus 0.02% TFA in 30 minutes. 76 mg of purified compound (11) was obtained with yield of 28%.

1H NMR (DMSO-d6) δ 0.820-1.155 (15H, m), 1.471-1.483 (4H, m), 1.931-1.919 (3H, m), 2.173-2.312 (1H, m), 2.602-2.979 (2H, m), 3.314-3.462 (1H, m), 4.101-4.161 (2H, m), 4.334-4.475 (1H, m), 4.538-4.624 (1H, m), 6.680-6.869 (1H, m), 6.901-6.999 (1H, m), 8.549-8.660 (1H, m).

Mass spectrum: calculated for C₂₂H₃₉N₅O₆: 469.57. MS: m/z 470.27 (M+1).

Analytical Calculation for C₂₂H₃₉N₅O₆.3TFA: C, 41.43; H, 5.22; N, 8.63.

Found by elemental analysis: C, 41.70; H, 5.42; N, 8.91.

### Example 3

### Preparation of a Benzyl-Val Analog of 4-Guanidinio Oseltamivir Carboxylate (GOC-Benzyl-Val)

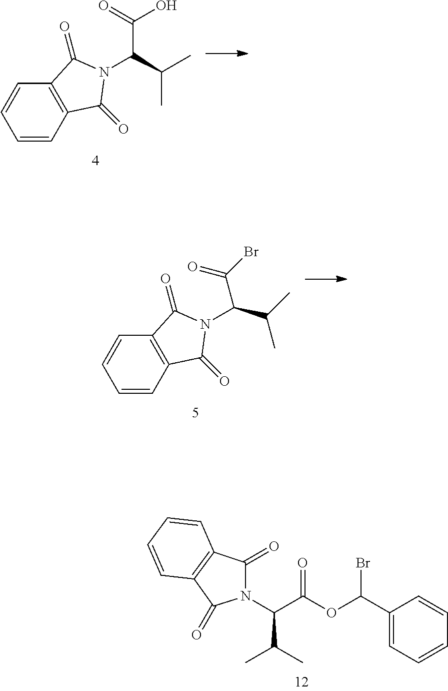

Compound 12:

13 ml of 1M oxalyl bromide in Dichloromethane (DCM) was added to a solution of 5 g phthaloyl valine (4) in 20 ml anhydrous DCM. After 78 uL of anhydrous dimethylformamide was added, the reaction was stirred overnight until bubbling ceased. The volatile components were removed by evaporation under argon. The residue (5) was re-dissolved in 10 ml anhydrous DCM and mixed with catalytic amount of anhydrous zinc chloride. After the temperature was lowered to −10° C. with ice-salt-water bath, 1.90 g benzaldehyde was added dropwise in 30 minutes. The reaction mixture was stirred at −5 to 5° C. for another 4 hours. The volatile components were removed by vacuum evaporation. The residue was subjected to a 100 g silica gel flash chromatography with 3:2 Hexane and ethyl acetate as eluent. 1.10 g of purified compound (12) was obtained with a yield of 13%.

1H NMR (DMSO-d6) δ 0.913-0.949 (3H, t), 1.148-1.161 (3H, d), 2.769-2.850 (1H, m), 4.571-4.643 (1H, m), 7.132 (1H, s), 7.250-7.844 (7H, m), 7.871-7.925 (2H, m).

Mass spectrum: calculated for C₂₀H₁₈BrNO₄: 416.27. MS: m/z 439.40 (M+Na⁺).

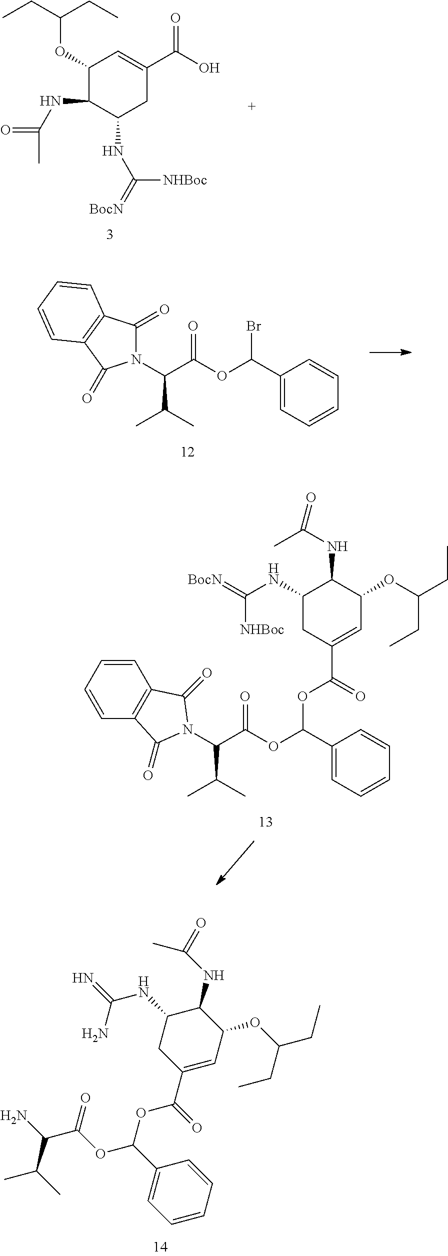

Compound 13:

0.3 ml of triethylamine was added in one portion to solution of 0.5 g of compound (3) and compound 1.0 g compound (12) in 20 ml anhydrous acetonitrile. The solution was heated at 80° C., stirred and refluxed for 3 hours before all of the volatile components were removed by vacuum evaporation. The residue was subjected to a 100 g silica gel flash chromatography with 1:1 Hexane and ethyl acetate as eluent. 300 mg of purified compound (13) was obtained with a yield of 36%.

1H NMR (DMSO-d6) δ 0.901-1.060 (12H, m), 1.105-1.173 (3H, m) 1.472-1.537 (22H, m), 2.176-2.401 (1H, m), 2.630-3.001 (2H, m), 3.341-3.468 (1H, m), 3.974-4.162 (2H, m), 4.316-4.452 (1H, m), 4.535-4.619 (1H, m), 6.179-6.257 (1H, m), 6.713-6.896 (1H, m), 7.045 (1H, s), 7.149-7.801 (7H, m), 7.834-7.906 (2H, m), 8.552-8.646 (1H, m), 11.376-11.411 (1H, m).

Mass spectrum: calculated for C₄₅H₅₉N₅O₁₂: 861.98. MS: m/z 863.01 (M+1).

Compound 14:

4.67 ml of 0.3M hydrazine monohydrate in absolute ethanol was added to a solution of 250 mg of compound (13) in 2 ml absolute ethanol. After stirring at room temperature for one hour, 5 ml trifluoroacetic acid was added and the solution was stirred for another 4 hours. After removal of all volatile components by vacuum evaporation, the residue was subjected to a 100 g reverse phase silica gel flash column with dichloromethane and Methanol (9:1 to 8:2). The collected sample was then further purified by reverse phase preparative HPLC with eluent of acetonitrile in water in a gradient method of 0% to 90% plus 0.02% TFA in 30 minutes. 51 mg of purified compound (14) was obtained with a yield of 34%.

1H NMR (D₂O) δ 0.879-1.054 (12H, m), 1.116-1.187 (3H, m) 1.452-1.476 (4H, m), 2.160-2.413 (1H, m), 2.625-3.108 (2H, m), 3.334-3.458 (1H, m), 3.994-4.181 (2H, m), 4.329-4.461 (1H, m), 4.516-4.600 (1H, m), 6.742-6.915 (1H, br), 7.044 (1H, s), 7.249-7.800 (5H, m), 8.536-8.650 (1H, m).

Mass spectrum: calculated for C₂₇H₄₁N₅O₆: 531.64. MS: m/z 532.70 (M+1).

Analytical calculation for C₂₇H₄₁N₅O₆.3TFA: C, 45.36; H, 5.08; N, 8.02. Found by elemental analysis: C, 45.30; H, 5.26; N, 8.15.

### Example 4

### Alternate Preparation of GOC-ISP-Valine

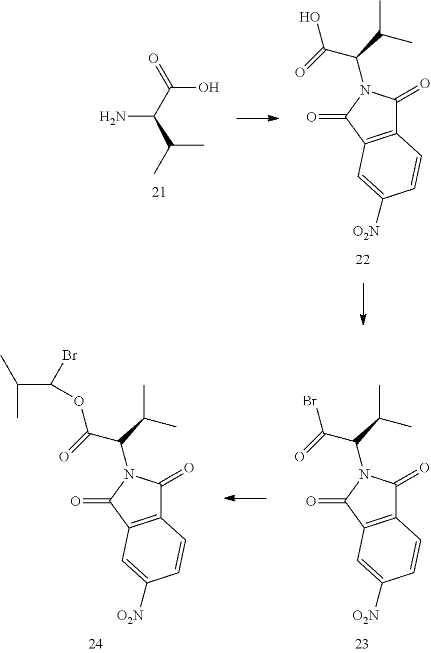

Compound 22:

1.8 ml of triethylamine was added slowly to a mixture of 15.1 g valine and 25 g 4-nitro-phthalic anhydride in 200 ml anhydrous toluene. The mixture was heated to reflux and stirred for three and half hours, during which time 1.8 ml of water was removed by toluene to a Dean-Stark water collector apparatus. After the reaction system cooled to room temperature, all volatile components were removed by vacuum evaporation. 32 g crude compound (22) was obtained.

5.73 g crude compound 22 were dissolved in minimum amount of dichloromethane and absorbed by 6 g of silica gel. The mixture was subjected to a 100 g of silica gel with 1.7 L 2:1 hexane/ethylacetate, as eluent. 4.93 g purified compound 22 was obtained from 5.73 g crude (73% yield from compound 21).

1H NMR (DMSO-d6) δ 0.923-0.940 (3H, d), 1.171-1.188 (3H, d), 2.714-2.802 (1H, m), 4.667-4.688 (1H, d), 8.073-8.082 (1H, d), 8.621-8.689 (2H, m).

Mass spectrum: calculated for C₁₃H₁₂N₂O₆: 292.24. MS: m/z 293.87 (M+1).

Compound 24:

10 ml of 1M oxalyl bromide in dichloromethane (DCM) was added to a solution of 4.9 g compound (22) in 20 ml anhydrous DCM. After 65 uL of anhydrous dimethylformamide was added, the reaction was stirred overnight until bubbling ceased. The volatile components were removed by evaporation under argon. The residue (23) was re-dissolved in 10 ml anhydrous DCM and mixed with catalytical amount of anhydrous zinc chloride. After the temperature was lowered to −10° C. with ice-salt-water bath, 1.53 g isobutyraldehyde was added dropwise in 30 minutes. The reaction mixture was stirred at −5 to 5° C. for another 4 hours. The volatile components were removed by vacuum evaporation. The residue was subjected to a 100 g silica gel flash chromatography with 3:2 Hexane and ethyl acetate as eluent. 1.29 g of purified compound (6) was obtained with yield of 35%.

1H NMR (DMSO-d6) δ 0.881-1.108 (12H, m), 2.014-2.102 (1H, m), 2.802-2.865 (1H, m), 4.641-4.685 (1H, m), 6.531-6.581 (1H, 2d), 8.065-8.103 (1H, d), 8.624-8.691 (1H, m), 8.704-8.712 (1H, m).

Mass spectrum: calculated for C₁₇H₁₉BrN₂O₆: 427.25. MS: m/z 450.10 (M+Na⁺).

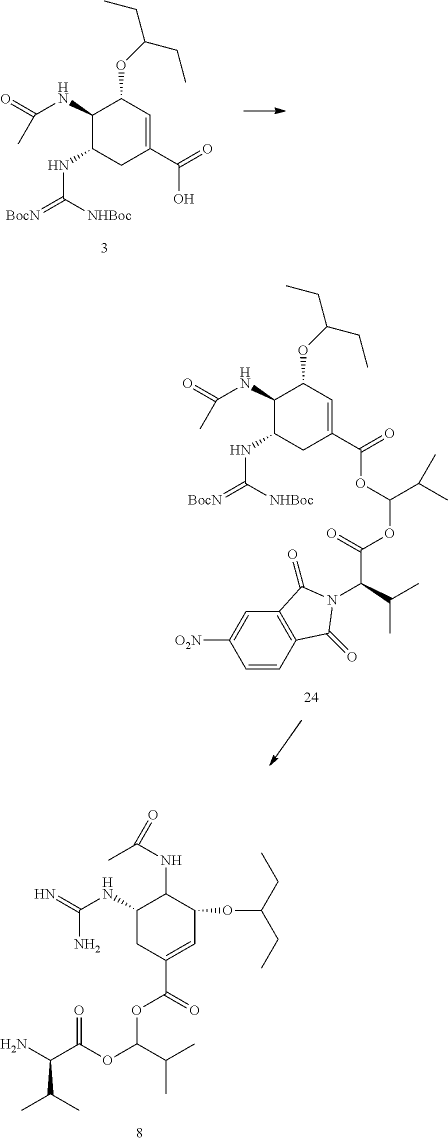

Compound 24:

0.3 ml triethylamine was added in one portion to solution of 0.5 g of compound 3 and 1.0 g compound 24 in 20 ml anhydrous acetonitrile. The solution was heated at 80° C., stirred and refluxed for 3 hours before all of the volatile components were removed by vacuum evaporation. The residue was subjected to a 100 g silica gel flash chromatography with 1:1 Hexane and ethyl acetate as eluent. 240 mg of purified compound 13 was obtained with a yield of 40%.

1H NMR (DMSO-d6) δ 0.821-0.843 (6H, m), 0.895-1.107 (12H, m) 1.446-1.508 (22H, m), 1.890-1.921 (3H, m), 2.236-2.315 (1H, m), 2.646-2.800 (2H, m), 3.300-3.371 (1H, m), 4.089-4.158 (2H, m), 4.310-4.430 (1H, m), 4.568-4.621 (1H, m), 5.301 (1H, s), 6.218-6.270 (1H, m), 6.693-6.732 (1H, m), 6.834-6.859.

(1H, m), 8.073-8.110 (1H, m), 8.631-8.714 (3H, m), 11.373 (1H, s).

Mass spectrum: calculated for C₄₂H₆₀N₆O₁₄: 872.96. MS: m/z 874.07 (M+1).

Compound 8:

4.67 ml of 0.3M monomethylhydrazine in absolute ethanol was added to a solution of 200 mg of compound (24) in 2 ml absolute ethanol. After stirring at room temperature for one hour, ml trifluoroacetic acid was added and the solution was stirred for another 4 hours. After removal of all volatile components by vacuum evaporation, the residue was subjected to a 100 g reverse phase silica gel flash column with dichloromethane and methanol (9:1 to 8:2). The collected sample was then further purified by reverse phase preparative HPLC with eluent of acetonitrile in water in a gradient method of 0% to 90% plus 0.02% TFA in 30 minutes. 63 mg of purified compound (25) was obtained with yield of 54%.

All analytical data for compound (8) in example 4 were the same as those for the compound (8) synthesized by phthaloyl protection method that was described in example 1 scheme 2.

### Example 5

### Alternate Preparation of GOC-ISP-Valine

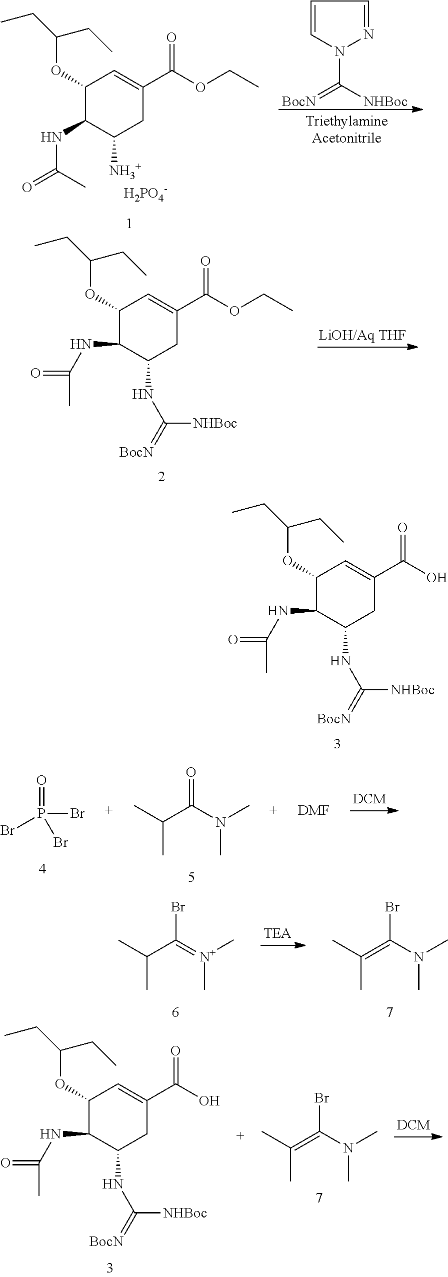

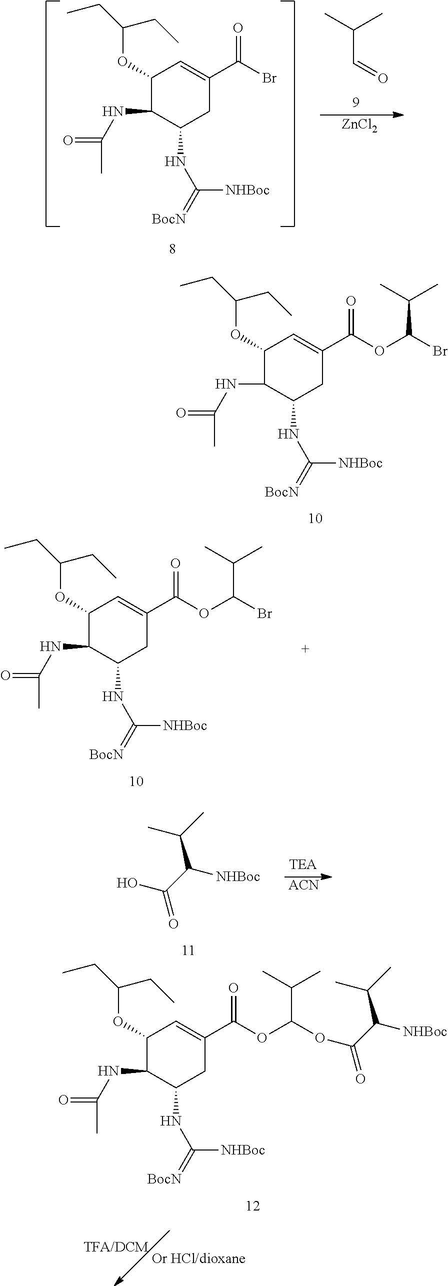

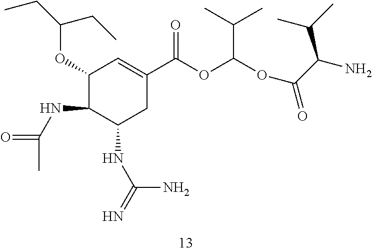 [SMILES: CCC(CC)O[C@@H]1C=C(C(=O)OC(OC(=O)[C@H](N)C(C)C)C(C)C)C[C@H](NC(=N)N)[C@H]1NC(C)=O]

Compound 2:

1.13 g (3.65 mmole) N,N′-bis-Boc-1-Guanylpyrazole was added to a suspension of 1.5 g (3.65 mmole) oseltamivir monophosphate (compound 1) in 20 ml anhydrous acetonitrile. After addition of 1.2 ml (8.7 mmole) of triethyl amine, the suspension was stirred at room temperature for 18 hours. All volatile components were removed by vacuum. The residue was purified by 100 g silica gel flash chromatography. 2 g of purified compound 2 was obtained with yield of 98%.

1H NMR (DMSO-d6) δ 0.787-0.864 (6H, m), 1.216-1.251 (3H, t), 1.355-1.396 (22H, m), 1.799 (3H, s), 2.318-2.344 (1H, m), 2.659-2.672 (1H, m), 3.400-3.428 (1H, m), 3.959-4.057 (2H, m), 4.134-4.232 (3H, m), 6.661 (1H, s), 7.893-7.913 (1H, d), 8.541-8.561 (1H, d), 11.523 (1H, s)

Mass spectrum: calculated for C₂₇H₄₆N₄O₈: 554.68. MS: m/z 550.20 (M+1)

Compound 3: 8.5 ml 1.46M KOH aqueous solution was added to a solution of 1.74 g (3.1 mmole) compound 2 in 12 ml tetrahydrofuran and 4 ml methanol. The mixture was stirred at room temperature overnight. All volatile components were removed by vacuum. 200 ml 0.1M phosphate buffer at pH of 6 was added to the white solid and, after stirring for 10 minutes, 0.1M potassium bisulfate was added dropwise carefully to adjust the pH to around 4.5 at which point a white precipitate was formed. 200 ml dichloromethane was added to dissolve all precipitate. The mixture was transferred to a separatory funnel and the dichloromethane layer was separated and washed with 100 ml water and 100 ml brine. The organic layer was dried over anhydrous sodium sulfate and the dichloromethane solvent was removed by vacuum. 1.32 g compound 3 was obtained with 80% yield.

1H NMR (DMSO-d6) δ 0.787-0.862 (6H, m), 1.397-1.494 (22H, m), 1.800 (3H, s), 2.234-2.295 (1H, m), 3.389-3.417 (1H, m), 3.946-4.057 (2H, m), 4.170-4.202 (1H, m), 6.714 (1H, s), 7.882-7.902 (1H, d), 8.530-8.549 (1H, d), 11.446 (1H, s), 12.700 (1H, br)

Mass spectrum: calculated for C₂₅H₄₂N₄O₈: 526.62. MS: m/z 527.20 (M+1)

Compound 7: * The preparation of bromoenamine (7) is described in literature: Leon Ghosez, etc, A general and practical method of synthesis of 2-disubstituted-1-chloro- and 1-bromoenamines, Tetrahedron 1998 (54) 9207-9222

Compound 10: To a solution of a compound 3 in dry DCM, bromoenamine (7) is added and the solution stirred under argon for 15 min; the total conversion of the acids to bromides is checked by TLC after quenching with MeOH. When the conversion is complete, all volatile components are removed at high vacuum under protection of argon. The residue is re-dissolved in anhydrous dichloromethane. 1M ZnCl2 in diethyl ether is added while the mixture is cooled in −10° C. ice-salt-water bath. Isobutyraldehyde is then added dropwise in a period of half hour while temperature should be controlled at −5 to 0° C. The reaction mixture is keep stirring at 0° C. for another 4 hours and at room temperature overnight. All volatile components were removed by vacuum. The residue is subjected to flash chromatography to obtain compound 10 with 68% yield.

1H NMR (CDCl3) δ 0.78-0.86 (6H, m), 0.89-1.07 (6H, m) 1.41-1.49 (22H, m), 1.80 (3H, s), 2.16-2.22 (1H, m), 2.23-2.33 (1H, m), 3.39-3.40 (1H, m), 3.96-4.01 (2H, m), 4.17-4.21 (1H, m), 6.30-6.33 (1H, d) 6.71 (1H, s), 7.88-7.91 (1H, d), 8.53-8.56 (1H, d), 11.44 (1H, s), 12.845 (1H, br)

Mass spectrum: calculated for C₂₉H₄₉BrN₄O₈: 661.63. MS: m/z 663.01 (M+1)

Compound 13: Compound (10) is dissolved in 5 ml anhydrous acetonitrile. Re-distilled triethylamine and Nα-Boc-valine-OH (11) are added. The mixtures are refluxed in oil bath for 4 hours. Volatile components are removed and the residue is purified by flash silica gel chromatography with eluent of 1:1 Hexan/EtOAc (v/v) to obtain (12). The compound (20) is dissolved in mixture of 4:1 DCM and TFA. After stirring for 4 hours, volatile components are removed by rotavapor and the residue is freeze dried to obtain (13) with 35% yield from (10).

1H NMR (D₂O) 0.78-1.07 (18H, m), 1.52 (4H, m), 1.80-1.82 (3H, m), 2.16-2.29 (3H, m), 3.385-3.401 (1H, m), 3.95-4.07 (2H, m), 4.17-4.20 (1H, m), 4.22-4.30 (1H, t), 5.305 (1H, s), 6.294-6.335 (1H, d) 6.714 (1H, s), 8.530-8.549 (1H, m)

Mass spectrum: calculated for C₂₄H₄₃N₅O₆: 497.63. MS: m/z 498.27 (M+1)

Analytical Calculation for C₂₄H₄₃N₅O₆.3TFA: C, 42.92; H, 5.52; N, 8.34. Found by elemental analysis: C, 42.85; H, 5.77; N, 8.17.

Total yield of compound 13 is 19% from compound 1 (oseltamivir monophosphate)

Compounds of formula (III) may be prepared by a number of synthetic routes. One such route is outlined in the following scheme.

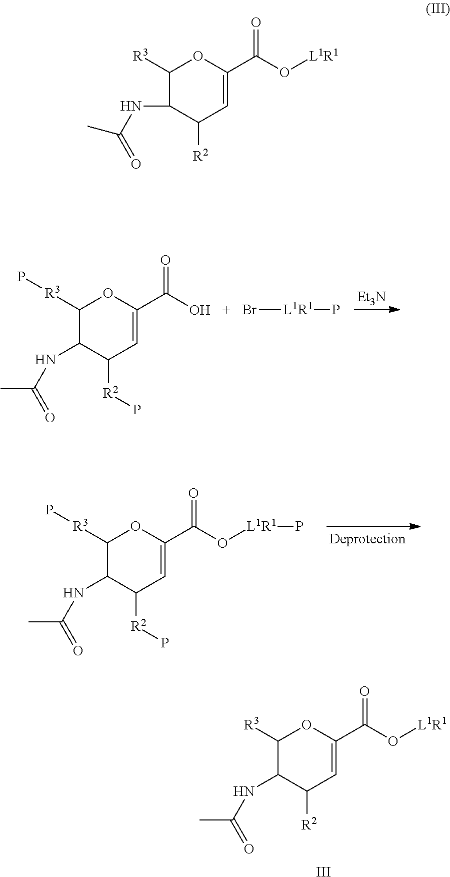

wherein P is a protecting group;
and L′, R¹, R², and R³ are as defined for formula (I).

### Example 6

### Synthesis of a Isopropyl-Valine Analog of Zanamivir (ZAN-Isp-Val)

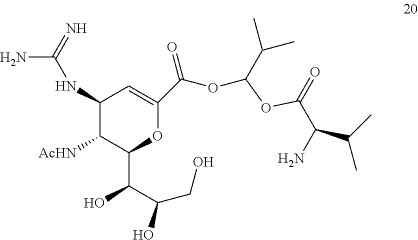 [SMILES: CC(=O)N[C@H]1[C@H]([C@H](O)[C@H](O)CO)OC(C(=O)OC(OC(=O)[C@H](N)C(C)C)C(C)C)=C[C@@H]1NC(=N)N]

The ZAN-Isp-Val was prepared according to the following procedure:

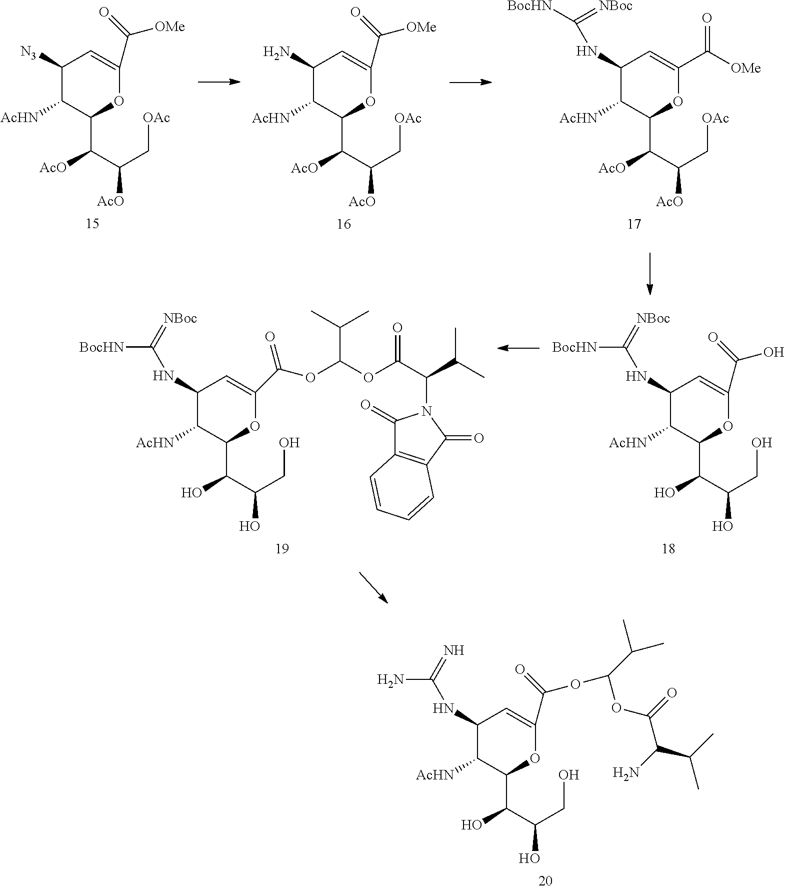

Compound 15:

Compound (15) was prepared according to existing method “Chandler, M.; Bamford, M. J.; Conroy, R. et al., J. CHEM. SOC. PERKIN TRANS. 1 (1995) 1173-1180.”

Compound 16:

540 mg of 10% Pd/C was added to a solution of 3 g compound (15) in 57 ml methanol, 35 ml toluene and 10 ml acetic acid. After evacuation of air, hydrogen was added to the reaction apparatus through a balloon. The mixture was stirred for 1 hour before all volatile components were removed by vacuum evaporation. The residue was re-dissolved in methanol and filtered to remove Pd/C. After removal of methanol by evaporation, the residue was subjected to a 60 g silica gel flash chromatography with 5:2:1 ethyl acetate/2-propanol/water as eluent. 1.8 g of purified compound (16) was obtained with yield of 65%.

1H NMR (DMSO-d6) δ 1.764 (3H, s), 1.990 (9H, s), 3.693 (3H, s), 3.700-3.798 (1H, m), 4.032-4.151 (2H, m), 4.226 (2H, br), 4.436-4.498 (2H, m), 5.174-5.251 (1H, m), 5.274-5.346 (1H, m), 5.321 (1H, d), 7.765-7.789 (1H, d).

Mass spectrum: calculated for C₁₈H₂₆N₂O₁₀: 430.41. MS: m/z 431.20 (M+1).

Compound 17:

1.23 g N,N′-bis-Boc-1-Guanylpyrazole was added to a solution of 1.7 g compound (16) in 20 ml anhydrous acetonitrile. After addition of 0.7 ml of triethyl amine, the solution was stirred at room temperature for 18 hours. All volatile components were removed by vacuum evaporation. The residue was purified by 100 g silica gel flash chromatography with eluent of 2:1 Ethylacetate/Hexane. 2.12 g of purified compound (17) was obtained with yield of 80%.

1H NMR (DMSO-d6) δ 1.403 (9H, s), 1.459 (9H, s), 1.722 (3H, s), 1.990-1.997 (9H, s), 3.710 (3H, s), 4.005-4.097 (2H, m), 4.394-4.429 (2H, m), 4.765-4.815 (1H, m), 5.225-5.266 (1H, m), 5.332-5.353 (1H, m), 5.845-5.850 (1H, d), 8.007-8.031 (1H, d), 8.141-8.160 (1H, d), 11.370 (1H, s).

Mass spectrum: calculated for C₂₉H₄₄N₄O₁₄: 672.68. MS: m/z 673.70 (M+1).

Compound 18:

3 ml of 1N sodium hydroxide aqueous solution was added at 0° C. to a solution of 1.2 g compound (17) in 10 ml tetrahydrofuran. The mixture was stirred at room temperature overnight followed by dryness with vacuum evaporation. 200 ml 0.1M phosphate buffer at pH of 6 was added to the white solid. After stirring for 10 minutes, 0.1M potassium bisulfate was added dropwise carefully to adjust the pH to around 4.5 at which point a white precipitate was formed. 200 ml dichloromethane was added to dissolve all precipitate. The mixture was transferred to a separatory funnel. The dichloromethane layer were separated and washed with 100 ml water and 100 ml brine. After drying the solution over anhydrous sodium sulfate, the dichloromethane solvent was removed by vacuum evaporation. 0.79 g compound (18) was obtained with 82% yield.

1H NMR (DMSO-d6) δ 1.410 (9H, s), 1.467 (9H, s), 1.801 (3H, s), 3.368-3.452 (3H, m), 3.621-3.669 (3H, m), 3.954-4.073 (3H, m), 4.712-4.760 (1H, m), 5.472-5.477 (1H, d), 8.148-8.317 (2H, m), 11.419 (1H, s).

Mass spectrum: calculated for C₂₂H₃₆N₄O₁₁: 532.54. MS: m/z 533.07 (M+1).

Compound 19:

0.4 ml of triethylamine was added in one portion to solution of 0.7 g of compound (18) and 1.64 g of compound (6) in 20 ml anhydrous acetonitrile. The solution was heated at 80° C., stirred and refluxed for 3 hours before all of the volatile components were removed by vacuum evaporation. The residue was subjected to a 100 g silica gel flash chromatography with 1:1 Hexane and ethyl acetate as eluent. 260 mg of purified compound (19) was obtained with a yield of 25.4%.

1H NMR (DMSO-d6) δ 0.830-0.912 (12H, m), 1.408-1.435 (18H, two singlet), 1.824 (3H, s), 2.540-2.606 (1H, m), 3.370-3.450 (3H, m), 3.624-3.675 (3H, m), 3.959-4.063 (3H, m), 4.589-4.593 (1H, m), 4.732-4.774 (1H, m), 5.469-5.470 (1H, d), 6.679-6.791 (1H, m), 7.768-7.896 (4H, m), 8.150-8.324 (2H, m), 8.596-8.641 (1H, m), 11.430 (1H, s).

Mass spectrum: calculated for C₃₉H₅₅N₅O₁₅: 833.88. MS: m/z 834.80 (M+1).

Compound 20:

4.67 ml of 0.3M hydrazine monohydrate in absolute ethanol was added to a solution of 230 mg of compound (19) in 2 ml absolute ethanol. After stirring at room temperature for one hour, 5 ml trifluoroacetic acid was added and the solution was stirred for another 4 hours. After removal of all volatile components by vacuum evaporation, the residue was subjected to a 100 g reverse phase silica gel flash column with dichloromethane and methanol (9:1 to 8:3). The collected sample was then further purified by reverse phase preparative HPLC with eluent of acetonitrile in water in a gradient method of 0% to 90% plus 0.02% TFA in 30 minutes. 68 mg of purified compound (20) was obtained with yield of 39%.

1H NMR (DMSO-d6) δ 0.908-1.024 (12H, m), 1.915 (3H, s), 2.001-2.082 (1H, m), 3.385-3.554 (3H, m), 3.621-3.671 (3H, m), 3.889-4.192 (3H, m), 4.554-4.595 (1H, m), 4.650-5.200 (1H, br), 5.487-5.492 (1H, d), 7.100-7.900 (6H, br), 7.904-7.926 (2H, m), 8.654-8.675 (1H, m).

Mass spectrum: calculated for C₂₁H₃₇N₅O₉. 503.55. MS: m/z 504.08 (M+1).

Anal. Calcd. For C₂₁H₃₇N₅O₉.3TFA: C, 38.35; H, 4.77; N, 8.28. Found: C, 38.41; H, 4.83; N, 8.32.

### Example 7

### General Scheme for Preparation of a Compound of Formula III

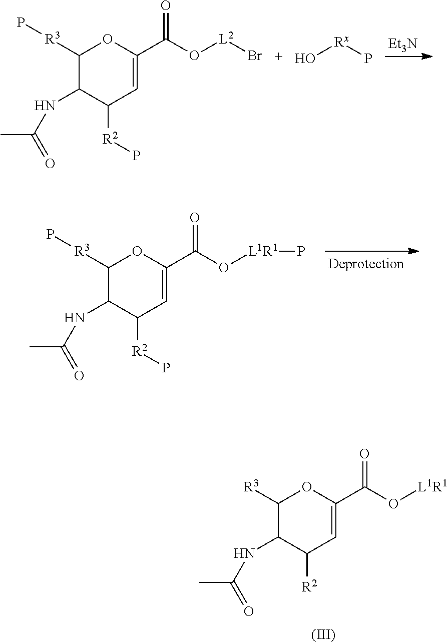

wherein P is a protecting group
L² is —(CR^{o}R^{o})ₘC(R⁴)₂(CR^{o}R^{o})ₙ—
R^{x} is —(CR^{o}R^{o})ₒ—C(O)(CR^{o}R^{o})ᵣC(R^{o}R′)(CR^{o}R^{o})ₛNH₂, —(CR^{o}R^{o})ₒ—C(O)(CR^{o}R^{o})ᵣC(R^{o}R′)(CR^{o}R^{o})ₛN(H)C(O)(CR^{o}R^{o})_{w}C(R^{o}R″)(CR^{o}R^{o})ₓNH₂, or —(CR^{o}R^{o})ₒC(O)(CR^{o}R^{o})ᵣC(R^{o}R′)(CR^{o}R^{o})ₛN(H)C(O)(CR^{o}R^{o})_{w}C(R^{o}R″)(CR^{o}R^{o})ₓN(H)C(O)(CR^{o}R^{o})_{y}C(R^{o}R′″)(CR^{o}R^{o})NH₂; and
L¹, R¹, R², R³, R⁴, R^{o}, R′, R″, m, n, o, r, s, w, x, y and z, are as defined for formula (I)

The ZAN-Isp-Val was prepared according to the following procedure:

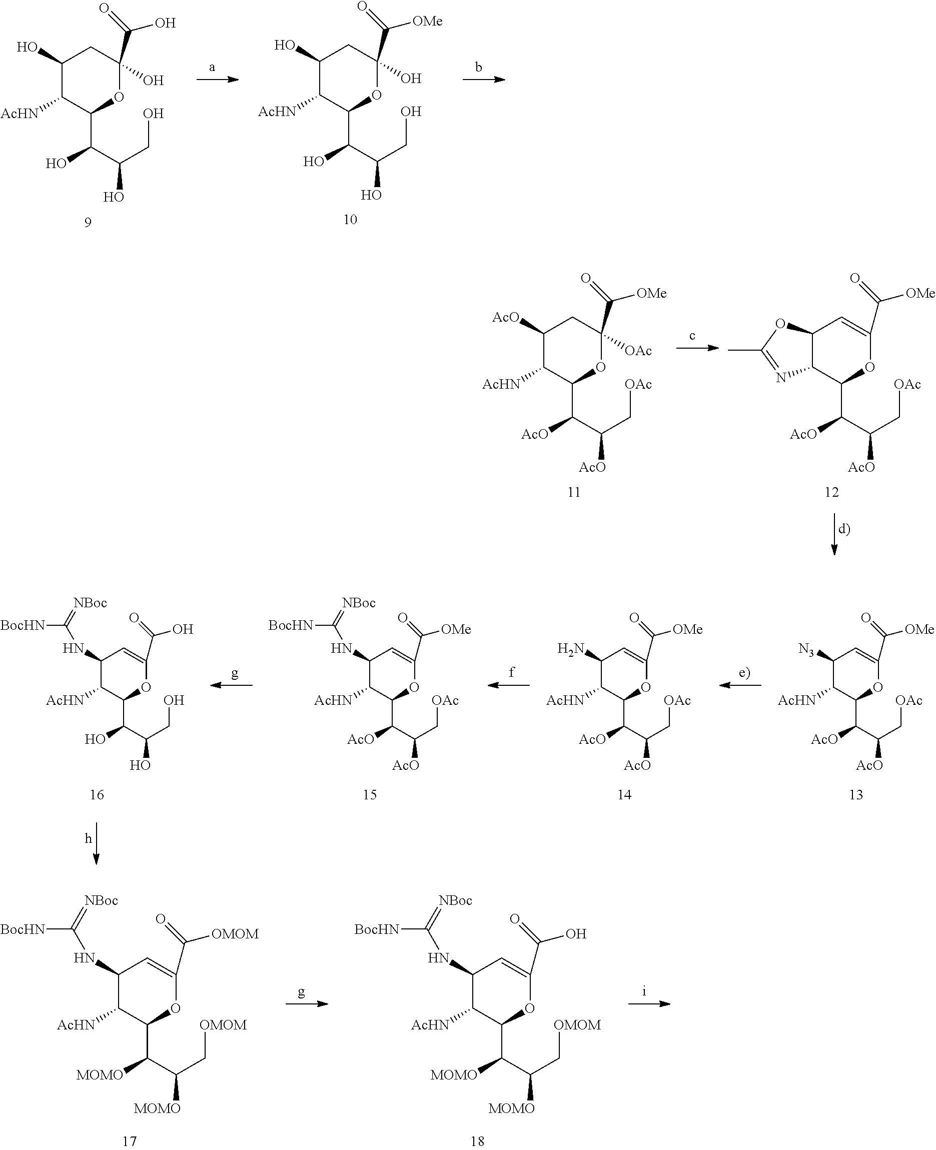

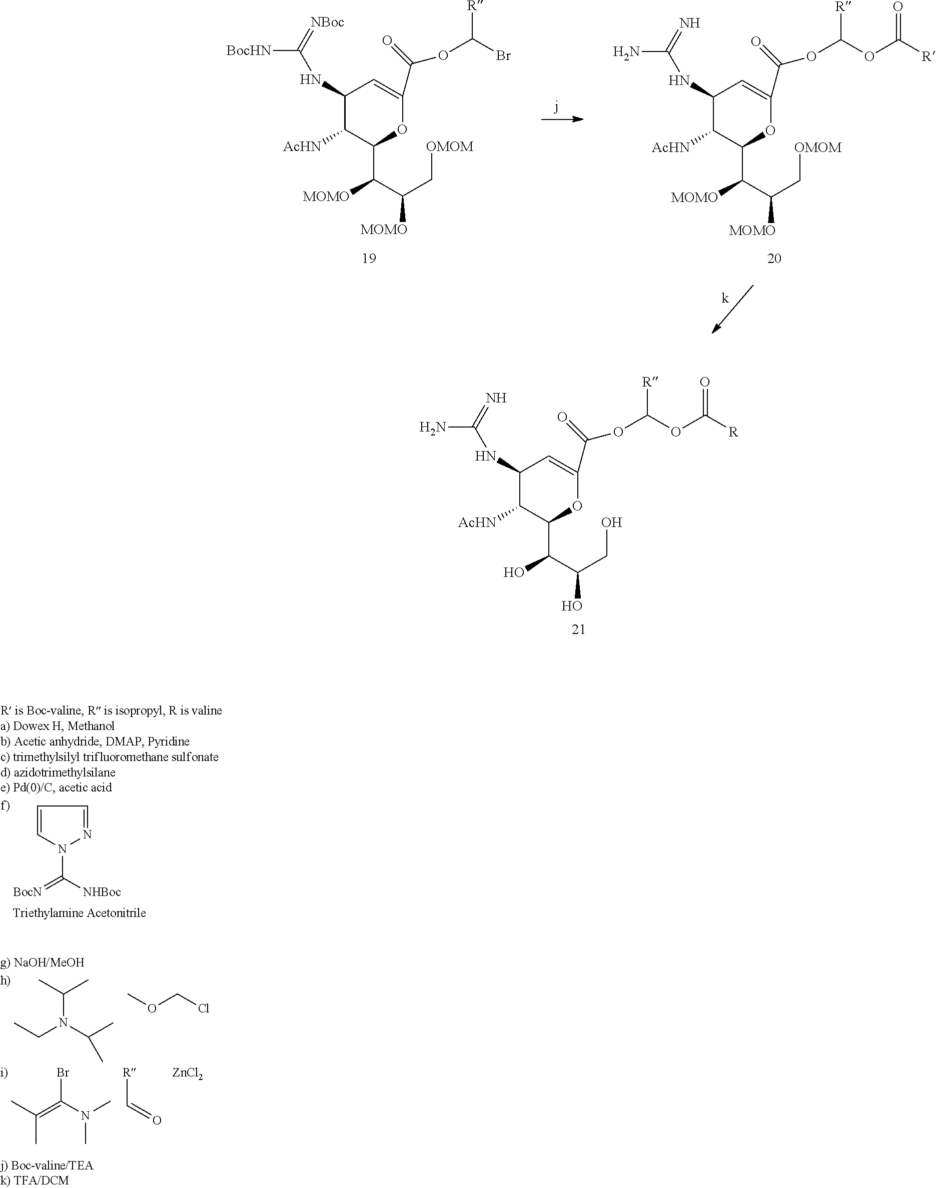

Compound 11:

Add Dowex-50 (H+) (10 g) to a suspension of N-acetyl neuraminic acid (9) (5 g, 0.16 mmol) in methanol and leave the mixture to stir at 40-45° C. overnight. In the course of the reaction the cloudy mixture will turn clear. Filter off the resin and rinse with methanol several times in order to collect the product sticking to the resin. The combined filtrate and washings are evaporated in vacuo and exposed to high vacuum overnight to obtained compound (10).

The compound (10) is suspended in 18 ml anhydrous pyridine. 15 ml (0.16 mmole) of acetic anhydride is added dropwise to the mixture that is cooled by external ice-water bath. Stir the mixture overnight at room temperature. The volatile components are evaporated by rotavapor. The residue is co-evaporated with toluene several times to remove extra pyridine, acetic anhydride and acetic acid. The resulting residue is dissolved in 100 ml ethyl acetate and washed with 100 ml 2N HCl aqueous and water respectively. The ethyl acetate solution is then washed with NaHCO₃ and brine, dried with Na₂SO₄. After removal of solvent, the residue is subjected to a flash silicon chromatography to obtain compound (11).

Compound 12:

Compound (11) (7.2 g) is dissolved in warm ethyl acetate (36 ml) and the solution is then cooled to 30° C. while TMSOTf (7.6 ml, 39 mmol) is then added dropwise during 10 min with stirring (magnetic stirrer) of the mixture under an inert atmosphere of argon. After the addition is complete the temperature is raised to 52° C. over a period of 20 min. After 2.5 h at this temperature the reaction mixture is allowed to cool and is poured into a vigorously stirred mixture of ice-cold saturated aq. sodium hydrogen carbonate (36 ml) and solid sodium hydrogen carbonate (10 g). Owing to the acid lability of the oxazoline, care is taken to ensure the solution remains basic (pH>7.5 as measured by universal indicator paper). After ca. 10 min the solution is filtered and the aqueous phase is separated and extracted with ethyl acetate (2×50 ml). The combined organic layers are concentrated to approximately half the original volume, and the resulting precipitate is removed and discarded by filtration. The filtrate is then evaporated to leave an amber gum. This is dissolved in hot propan-2-ol (10 ml) which, on cooling in an ice-water bath, deposit crystals. The mixture is filtered and the filter is washed with a mixture of diisopropyl ether and propan-2-ol (2:1) to give (12) after being dried in vacuo at 40° C. (3.44 g, 61.7%)

Compound 13:

A stirred solution of the oxazoline 12 (6 g, 14.5 mmol) in tert-butyl alcohol (4.5 ml) containing azidotrimethylsilane (2.89 ml, 21.8 mmol) under argon, is heated to reflux on a steam-bath. A hot-water condenser is used to prevent any possible condensation of hydrazoic acid. After 10.5 h the reaction mixture is allowed to cool overnight. Aqueous sodium nitrite (1.2 g in 6 ml water) is then added. 6 M hydrochloric acid is then added dropwise over a period of 1 h to give vigorous evolution of gases. Ethyl acetate (30 ml) and water (30 ml) are added and the organic layer is separated off and washed with water (2×50 ml). The combined aqueous layers are back-extracted with ethyl acetate (50 ml) and the combined organic layers are washed successively with 6% aq. sodium hydrogen carbonate (2×30 ml) followed by brine (30 ml). Aqueous residues are removed cautiously. The combined organic extracts are dried (MgSO₂), and evaporated under reduced pressure at 48-50° C. (rotary evaporator) to give an oil. This is dissolved in propan-1-ol (20 ml) and treated dropwise with water (20 ml) added over a period of 1 h. The resulting crystalline solid is filtered off, and washed with water (2×18 ml) to give compound (13) after being dried in high vacuum at 42° C. for 24 h (5.23 g, 76%).

Compound 14:

To a solution of compound 13 (1 g, 2.19 mmol) in MeOH (38 ml) is added toluene (23 ml), Pd—C (10%) (190 mg), and acetic acid (0.2 g, 3.33 mmol). This mixture is hydrogenated at atmospheric pressure for 1 h and then filtered. The filtrate is evaporated to dryness, and the residue is subjected to flash chromatography (silica gel, 5:2:1 EtOAc/2-propanol/water) to afford pure compound (14) (0.68 g, 72%).

Compound 15:

1.13 g (3.65 mmole) N,N′-bis-Boc-1-Guanylpyrazole is added to a suspension of 0.6 g (1.47 mmole) compound 14 in 20 ml anhydrous acetonitrile. After addition of 0.6 ml (4.3 mmole) of triethyl amine, the suspension 1 s stirred at room temperature for 18 hours. All volatile components are removed by vacuum. The residue is purified by 30 g silica gel flash chromatography.

Compound 16:

8.5 ml 1.46M NaOH aqueous solution is added to a solution of compound 15 in 12 ml tetrahydrofuran and 4 ml methanol. The mixture is stirred at room temperature overnight. All volatile components are removed by vacuum. 200 ml 0.1M phosphate buffer at pH of 6 is added to the white solid and, after stirring for 10 minutes, 0.1M potassium bisulfate is added dropwise carefully to adjust the pH to around 4.5 at which point a white precipitate is formed. 200 ml dichloromethane is added to dissolve all precipitate. The mixture is transferred to a separatory funnel and the dichloromethane layer is separated and washed with 100 ml water and 100 ml brine. The organic layer is dried over anhydrous sodium sulfate and the dichloromethane solvent is removed by vacuum to obtained compound (16).

Compound 17:

To a solution of the compound (16) in 1 ml of methylene chloride is added N,N-diisopropylethylamine and chloromethyl methyl ether. The reaction mixture then is refluxed for 5.5 h. After cooling to room temperature, the reaction mixture is diluted with ethyl acetate and is washed with 5% hydrochloric acid and brine. The organic layer is dried over anhydrous magnesium sulfate, and the solvent is removed under reduced pressure. The residue is then subjected to flash chromatography to obtain compound (17).

Compound 18:

A solution of compound (17) in 1.2 ml of methanol cooled to 0° C. is treated dropwise with 0.6 ml of 1N aqueous sodium hydroxide. After 1 h at 0° C., the reaction temperature is warmed to room temperature, where stirring is continued for an additional 20 h. All volatile components are removed by vacuum. 200 ml 0.1M phosphate buffer at pH of 6 is added to the white solid and, after stirring for 10 minutes, 0.1M potassium bisulfate is added dropwise carefully to adjust the pH to around 4.5 at which point a white precipitate is formed. 200 ml dichloromethane is added to dissolve all precipitate. The mixture is transferred to a separatory funnel and the dichloromethane layer is separated and washed with 100 ml water and 100 ml brine. The organic layer is dried over anhydrous sodium sulfate and the dichloromethane solvent is removed by vacuum to obtained compound (18).

Compound 19:

To a solution of a compound (18) in dry DCM, bromoenamine* is added and the solution stirred under argon for 15 min; the total conversion of the acids to bromides is checked by TLC after quenching with MeOH. When the conversion is complete, all volatile components are removed at high vacuum under protection of argon. The residue is re-dissolved in anhydrous dichloromethane. 1M ZnCl₂ in diethyl ether is added while the mixture is cooled in −10° C. ice-salt-water bath. Isobutyraldehyde is then added dropwise in a period of half hour while temperature should be controlled at −5 to 0° C. The reaction mixture is keep stirring at 0° C. for another 4 hours and at room temperature overnight. All volatile components are removed by vacuum. The residue is subjected to flash chromatography to obtain compound (19). * The preparation of bromoenamine is described in literature: Leon Ghosez, et al. A general and practical method of synthesis of 2-disubstituted-1-chloro- and 1-bromoenamines, Tetrahedron 1998 (54) 9207-9222.

Compound 21:

Compound (19) is dissolved in 5 ml anhydrous acetonitrile. Re-distilled triethylamine and Nα-Boc-valine-OH are added. The mixture is refluxed in oil bath for 4 hours. Volatile components are removed and the residue is purified by flash silica gel chromatography with eluent of 1:1 Hexan/EtOAc (v/v) to obtain compound 20. The compound (20) is dissolved in mixture of 4:1 DCM and TFA. After stirring for 4 hours, volatile components are removed by rotavapor and the residue is freeze dried to obtain compound (21).

Compounds of formula IV may be prepared by a number of synthetic routes. One such route is outlined in the following scheme:

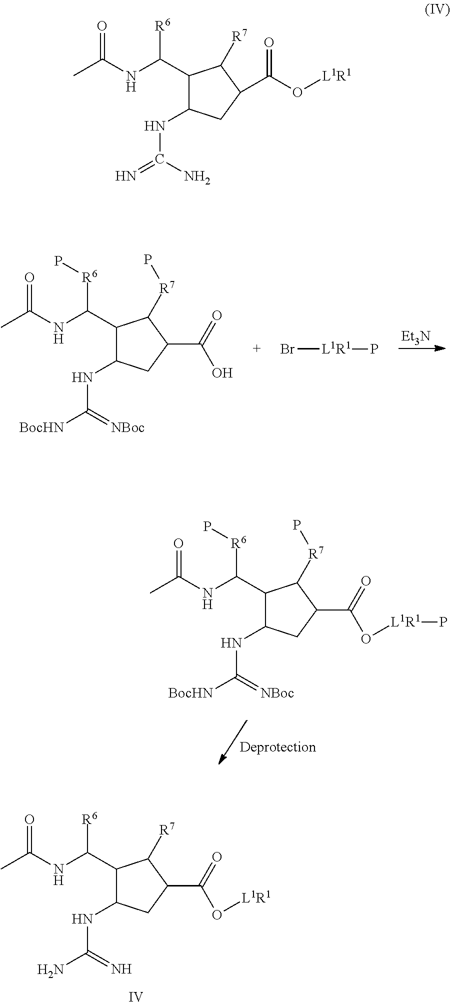

P is a protecting group;
and L¹, R¹, R⁶ and R⁷ are as defined in Formula (IV).

### Example 8

### Bioavailability of GOC and GOC Analogs after Oral Administration to Mice

GOC and analogs of GOC, GOC-Isp-Val, GOC-methyl-VAL and GOC-benzyl-VAL, were evaluated for oral bioavailability in mice in both the fasted and fed states.

Eight groups of mice (n=5 mice per group) as described in Table 1, were administered GOC or a GOC analog orally at a dose of ˜10 mg/kg in fasted and fed mice. Blood samples were taken at 0, 1, 2, 3, 4, 8, 12, 16 and 24 hours by heart stick. In separate experiments, mice were dosed intravenously with 1 mg/kg GOC and blood samples were taken at 0, 5, 10, 15, 30, 60, 120, 180 and 240 minutes via heart stick. All plasma samples were analyzed by LC/MS/MS. After administration of the analog, only the GOC was detectable in plasma. From the concentration versus time data, the AUC was calculated using the trapezoidal rule.

Results from the experiments are shown in Table 1. The bioavailability of GOC was 4.3% and 6% in the fasted and fed state respectively. In contrast, the bioavailability of GOC after oral administration of the GOC analogs was significantly greater than after oral administration of GOC. GOC-Isopropyl Valine showed 48.1% and 57.2% bioavailability in both the fasted and fed state, respectively. GOC-Methyl Valine showed 43.9% and 22.9% bioavailability in both the fasted and fed state, respectively. GOC-Benzyl Valine showed 13.5% and 12.1% bioavailability in both the fasted and fed state, respectively. In Table 1, Tₘₐₓ is the time to reach maximal concentration after dosing. Cₘₐₓ is the maximal concentration after dosing. AUC means area under the curve. T½ is the period of time required for the concentration of drug in plasma until concentration is exactly one-half of a given concentration. CL is the volume of blood from which all of a drug would appear to be removed per unit time. Vz means the volume of distribution. Bioavailability (BA) is calculated by the formula (AUC oral/AUC iv)×(dose of iv/dose of oral).

TABLE 1

| | Dosing | Tₘₐₓ | Cₘₐₓ | AUC | T_{1/2} | CL | Vz | BA |
|---|---|---|---|---|---|---|---|---|
| Compound | Route | (hrs) | (ng/ml) | (ng/ml * hrs) | (hr) | (ml/hr) | (ml) | (%) |
| GOC | IV | | | 541 ± 229 | 0.88 | 53 | 67.8 | |
| | PO (fasted) | 1.3 ± 1.0 | 43 ± 29 | 232 ± 43 | 1.21 | 2276 | 3961 | 4.3% |
| | PO (fed) | 3.0 ± 1.2 | 73 ± 30 | 327 ± 98 | 1.65 | 914 | 2181 | 6.0% |
| Isopropyl L-Val | IV | | | 226 ± 151 | 0.67 | 110 | 107 | |
| | PO (fasted) | 1.2 ± 0.4 | 249 ± 108 | 1086 ± 187 | 3.03 | 212 | 928 | 48.1% |
| | PO (fed) | 1.8 ± 1.3 | 317 ± 197 | 1292 ± 502 | 4.77 | 225 | 1549 | 57.2% |
| Methyl L-Val | IV | | | 730 ± 60 | 1.05 | 39 | 59.5 | |
| | PO (fasted) | 2.2 ± 1.1 | 561 ± 235 | 3205 ± 577 | 1.37 | 140 | 276 | 43.9% |
| | PO (fed) | 2.4 ± 1.3 | 444 ± 227 | 1669 ± 392 | 1.49 | 161 | 347 | 22.9% |
| Benzyl L-Val | IV | | | 867 ± 110 | 1.15 | 31 | 50.8 | |
| | PO (fasted) | 1.0 ± 0.0 | 192 ± 48 | 1171 ± 357 | 7.14 | 195 | 2014 | 13.5% |
| | PO (fed) | 1.6 ± 0.9 | 127 ± 69 | 1045 ± 449 | 9.49 | 270 | 3701 | 12.1% |

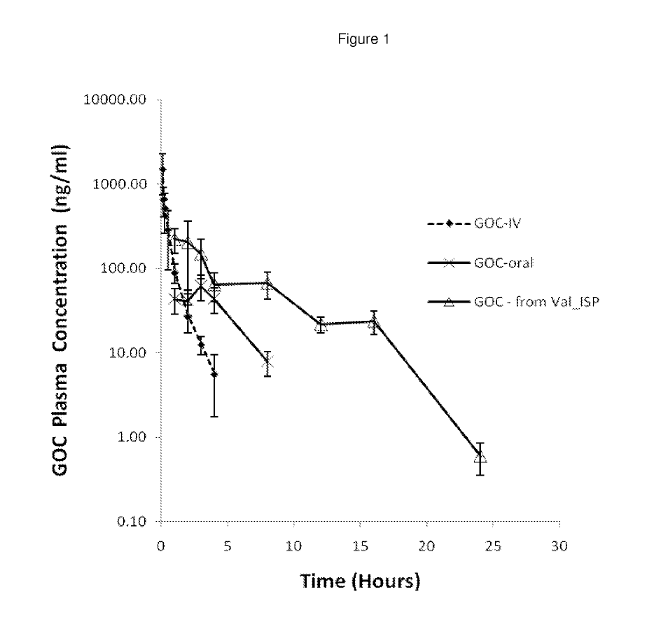

FIG. 1 shows a comparison of the GOC plasma levels after oral administration of 10 mg/kg of GOC-isopropyl Valine (Δ), GOC(X□) and IV administration of 1 mg/kg GOC (♦) to fed animals (n=5).

### Example 9

### Effects of GOC, Analogs of GOC, and Oseltamivir on Influenza a Virus Infection in Mice

Animals: Female 18-20 g BALB/c mice were obtained from Charles River Laboratories (Wilmington, Mass.) for this study. They were maintained on standard rodent chow and tap water ad libitum. The animals were quarantined for at least 48 hours prior to use.

Virus: Influenza A/NWS/33 (H1N1) was used. The virus was originally provided by Dr. Kenneth Cochran (University of Michigan, Ann Arbor). The virus had been passaged three times in mice and one time in MDCK cells. The virus pool was pre-titrated in mice prior to use in this experiment.

Compounds: The compounds were pre-weighed, and each tube of compound was hydrated just prior to oral gavage treatment of mice. Oseltamivir was purchased from a pharmacy. The compounds were prepared in sterile water.

Mice were anesthetized by intraperitoneal (i.p.) injection of ketamine/xylazine (50/5 mg/kg), and then exposed to virus intranasally with a 90-pt suspension of influenza virus. The infection inoculum of 10^{4.5} CCID₅₀/mouse (4 mouse LD₅₀) equated to a 100% lethal challenge dose in this experiment. Groups of mice were treated per oral with compounds twice a day (at 12 hour intervals) for 5 days starting 2 hours prior to virus exposure. Ten drug-treated infected mice and 20 placebo mice were observed daily for death through 21 days. Five additional uninfected mice injected with the highest (10 mg/kg/day) dose of each compound were maintained for the purpose of determining toxicity. Mice were weighed as a group every other day.

Statistical analysis: Initial comparisons of survival curves by Log-rank test were performed and it was found that the difference among groups was statistically significant (p<0.001). Pairwise comparisons of survivor numbers were then made using the two-tailed Fisher exact test. Differences in the mean day of death were statistically analyzed using the two-tailed Mann-Whitney U-test. All analyses were two-tailed and calculated using Prism and Instat software programs (GraphPad Software, San Diego, Calif.). Statistical comparisons were made between treated and placebo groups.

Results of treatment from the lethal infection are reported in Table 2. GOC was 100% protective at 10 mg/kg/day, but was not active at 1 and 0.1 mg/kg/day. GOC-Isp-Val was 100% protective at 0.1, 1, and 10 mg/kg/day. GOC-Me-Val was 100% protective at 10 mg/kg/day, 70% protective at 1 mg/kg/day, and inactive at 0.1 mg/kg/day. Oseltamivir was 100% protective at 1 and 10 mg/kg/day, but ineffective at 0.1 mg/kg/day. Thus, GOC-Isp-Val was the most potent of the four compounds tested (at least 10-fold more potent than oseltamivir).

TABLE 2

| Compound | Survivors/ | |
|---|---|---|
| (mg/kg/day) | Total | MDD^{a} ± SD |
| GOC (10) | 10/10** | — |
| GOC (1) | 1/10 | 9.8 ± 1.2 |
| GOC (0.1) | 0/10 | 9.0 ± 1.0 |
| GOC-Isp-Val (10) | 10/10** | — |
| GOC-Isp-Val (1) | 10/10** | — |
| GOC-Isp-Val (0.1) | 10/10** | — |
| GOC-Me-Val (10) | 10/10** | — |
| GOC-Me-Val (1) | 7/10** | 10.7 ± 0.6* |
| GOC-Me-Val (0.1) | 0/10 | 10.0 ± 1.3 |
| Oseltamivir (10) | 10/10** | — |
| Oseltamivir (1) | 10/10** | — |
| Oseltamivir (0.1) | 0/10 | 9.7 ± 1.6 |
| Placebo | 0/20 | 8.8 ± 0.8 |

^{a}Mean day of death of mice that died prior to day 21.
*P < 0.05,
**P < 0.001, compared to respective placebo.

Toxicity evaluations of compounds in uninfected mice are presented in Table 3. Slight weight loss was evident in all treated groups compared to normal controls, indicative of treatment stress. Weight loss was similar in all treated groups, and no deaths were reported, indicating that GOC and its analogs were not toxic to the mice relative to oseltamivir. The data are reported as weight loss in grams from initial body weight. The values in parenthesis are the % weight loss from initial body weights.

TABLE 3

| Compound | Survivors/ | Mean Weight | Mean Weight |
|---|---|---|---|
| (mg/kg/day) | Total | Change Day 5 | Change, day 7 |
| GOC (10) | 5/5 | −0.3 (−2.1) | −0.2 (−1.0) |
| GOC-Isp-Val (10) | 5/5 | −0.2 (−1.1) | 0.0 (0.0) |
| GOC-Me Valine (10) | 5/5 | −0.2 (−0.5) | −0.3 (−1.6) |
| Oseltamivir (10) | 5/5 | −0.3 (−1.6) | −0.1 (−0.5) |
| Placebo | 5/5 | −0.5 (−2.6) | −0.1 (−0.5) |
| Normal Control | 5/5 | +0.5 (+3.1) | +0.3 (+1.6) |

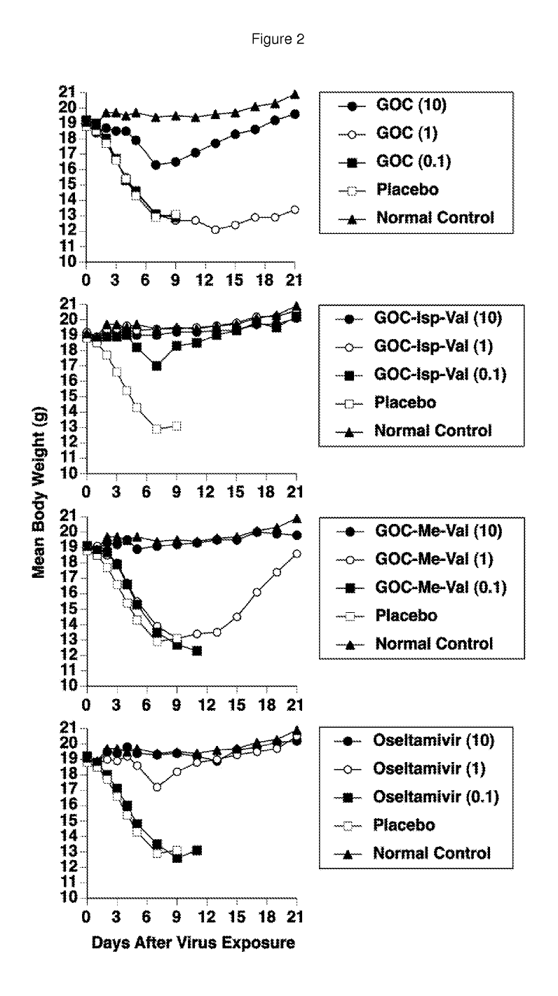

Body weights during the infection are reported in FIG. 2. The graphs show the extent of weight loss, and are useful for comparing groups exhibiting a high degree of survival. Some weight loss was seen in the group treated with 10 mg/kg/day of GOC. GOC-ISP-Valine (0.1 mg/kg/day and above) treatment resulted in minimal weight loss. Severe weight loss was evident in the 1 mg/kg/day GOC-Me-Val group, indicating that these mice barely survived the infection. The 1 mg/kg/day oseltamivir group lost minimal weight, which was similar to that of the 0.1 mg/kg/day GOC-ISP-Valine group (a 10-fold potency advantage for GOC-ISP-Valine).

### Example 10

### In Vitro Activity of GOC Versus Oseltamivir Carboxylate on Selected Influenza Virus

Virus strains: The viruses listed in Table 4 are recent clinical isolates and well known strains of virus. Madin Darby canine kidney (MDCK) cells were used to grow the virus.

Inhibition of Viral Cytopathic Effect (CPE): In the CPE inhibition test, cells are grown in 96 well flat-bottomed microplates. Four log 10 dilutions of each test compound (e.g. 1000, 100, 10, 1 μg/ml) were added to 3 wells containing the cell monolayer. Within 5 minutes the virus was added and the plate sealed, incubated at 37° C. for 3 to 4 days and the CPE was read microscopically. Neutral red is then added to the medium; cells not damaged by virus take up a greater amount of dye. The stained plate was is read on a computerized microplate autoreader. The method as described by McManus (Appl. Environment. Microbiol. 31:35-38, 1976) was used. The data from the stained cells are expressed as 50% effective concentrations (EC50).

Table 4 shows the in vitro activity of GOC versus Oseltamivir Carboxylate on Selected Influenza Viruses. Table 4 shows that the GOC is 10-fold to over 100-fold more potent than the oseltamivir carboxylate (OC). Of note is that the GOC remains active against the Oseltamivir resistant strain, Hong Kong/2369/2009 (H1N1)-H275Y and is 100-fold more active than OC against the H5N1 strain Duck/MN/1525/81.

TABLE 4

| | GOC | Oseltamivir |
|---|---|---|
| Virus | μM | Carboxylate μM |
| California/04/2009 (H1N1) | 0.016 | 0.3 |
| Hong Kong/2369/2009 (H1N1) | 1.7 | >100 |
| H275Y oseltamivir resistant | | |
| NWS/33 (H1N1) | <0.032 | 0.58 |
| Brisbane/10/2007 (H3N2) | >0.32 | >0.32 |
| Brisbane/59/2007 (H1N1) | 0.004 | >0.32 |
| Victoria/3/75 (H3N2) | 0.038 | >0.32 |
| Solomon Islands/03/2006 (H1N1) | 0.0042 | >0.32 |
| Sichuan/379/99 | >3.2 | >3.2 |
| Flu A Duck/MN/1525/81 (H5N1) | 0.0012 | 0.15 |
| Flu A Vietnam/1203/2004 (H5N1) × | 0.00028 | 0.0028 |
| A/Ann Arbor/6/60 (H1N1 core genes) | | |
| Flu B Florida/4/2006 | 0.46 | >3.2 |

### Example 11

### Zanamivir Plasma Levels after Oral Dosing of ZAN-Isp-Val

Eight groups of mice (n=5 mice per group) were administered ZAN-Isp-Val orally at a dose of ˜8 mg equivalents of Zanamivir/kg and blood samples were taken at 0, 1, 2, 3, 4, 8, and 24 hours by heart stick. In separate experiments, mice were dosed intravenously with 1 mg/kg Zanamivir and blood samples were taken at 0, 2, 5, 15, 30, 60 and 120 minutes via heart stick.

For the ZAN-Isp-Val dosing, plasma samples were analyzed by LC/MS/MS. After administration of the analog, only the Zanamivir was detectable in plasma.

For the Zanamivir IV dosing, the Zanamivir contained a radioactive tritium tracer. Aliquots of plasma were counted in a liquid scintillation counter. The counts were converted to ng of Zanamivir/ml of plasma through the following formula:

$$Zanamivir ( ng \text{/} ml of plasma ) = \frac{( cpm ) \times Specific Activity of dosing solution ( ng \text{/} cpm )}{aliquot size ( ml )} \qquad 1 )$$

From the concentration versus time data, the AUC was calculated using the trapezoidal rule. The bioavailability (% BA) was calculated by dividing the AUCₒᵣₐₗ by AUCᵢᵥ and normalizing the ratio for dose.

Table 5 shows the Plasma concentration of Zanamivir after dosing of either the ZAN-Isp-Val orally or Zanamivir by intravenous injection. These data indicate that ZAN-Isp-Val is completely absorbed in fasted animals after oral dosing.

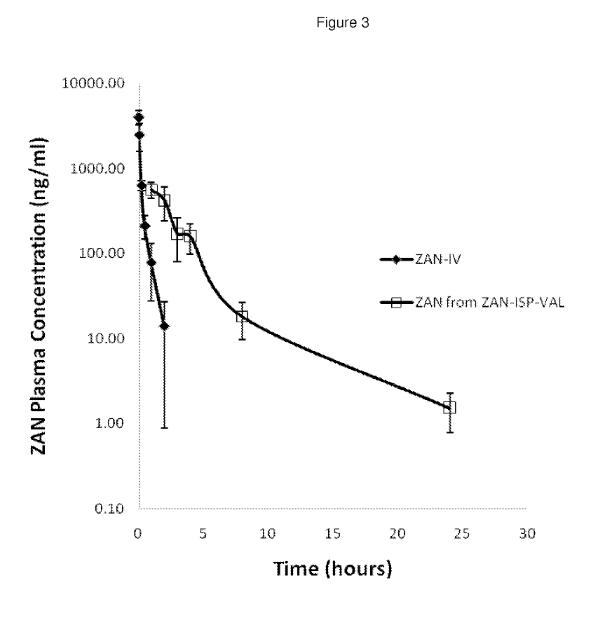

FIG. 3 shows The figure shows a comparison of the Zanamivir plasma levels after oral administration of 8.5 mg eq Zanamivir/kg of ZAN-Isp-Val (□) or IV administration of 1 mg/kg Zanamivir (♦) to fasted animals (n=5).

TABLE 5

| | |
|---|---|
| iv dose - | oral dose _ fasted |
| Zanamivir | ZAN-Isp-Val |

| Hours | ng/ml | stdev | ng/ml | std dev |
|---|---|---|---|---|
| 0.03 | 4018.3 | 786.9 | | |
| 0.08 | 2487.5 | 891.1 | | |
| 0.25 | 633.8 | 82.4 | | |
| 0.50 | 212.0 | 64.9 | | |
| 1.00 | 78.6 | 50.9 | 563.7 | 119.5 |
| 2.00 | 14.0 | 13.2 | 423.1 | 179.4 |
| 3.00 | | | 171.2 | 90.7 |
| 4.00 | | | 159.9 | 62.1 |
| 8.00 | | | 18.2 | 8.4 |
| 24.00 | | | 1.5 | 0.7 |

| | | Cmax | AUC | |
|---|---|---|---|---|
| Drug | Route | (ng/ml) | (ng/ml) × hr | % BA |
| ZAN-Isp-Val | Oral | 563.7 | 2177 | 100.3 |
| Zanamivir | IV | na | 202.4 | |

1. A compound of formula (I): 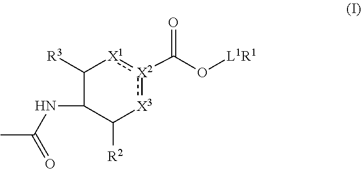
wherein: L¹ is —(CR^{o}R^{o})ₘC(R⁴)₂(CR^{o}R^{o})ₙO(CR^{o}R^{o})ₒ—;
R¹ is —C(O)(CR^{o}R^{o})ᵣC(R^{o}R′)(CR^{o}R^{o})ₛNH₂, —C(O)(CR^{o}R^{o})ᵣC(R^{o}R′)(CR^{o}R^{o})ₛN(H)C(O)(CR^{o}R^{o})_{w}C(R^{o}R″)(CR^{o}R^{o})ₓ—NH₂, or —C(O)(CR^{o}R^{o})C(R^{o}R′)(CR^{o}R^{o})ₛN(H)C(O)(CR^{o}R^{o})_{w}C(R^{o}R″)(CR^{o}R^{o})ₓN(H)C(O)(CR^{o}R^{o})_{y}C(R^{o}R″′)(CR^{o}R^{o})_{z}NH₂;
each occurrence of m, n, o, r, s, w, x, y, or z is independently zero, one, or two;
each occurrence of R^{o} is independently H, optionally substituted alkyl, optionally, substituted cycloalkyl, optionally substituted aryl, or optionally substituted heteroaryl;
R² is NH₂ or —NHC(NH₂)NH;
R³ is H, —OR*, or —CHR*R**;
R′, R″ and R′″ are each independently an amino acid side chain;
each occurrence of R⁴ is independently hydrogen or an optionally substituted group selected from a C₁-C₆ alkyl group, a 3-7 membered saturated, partially saturated or fully unsaturated monocyclic ring having 0-3 heteroatoms independently selected from nitrogen or oxygen or two occurrences of R⁴ are taken together with the atom(s) to which they are bound to form an optionally substituted 3-7 membered ring, wherein if one occurrence of R⁴ is H, then the other occurrence of R⁴ is not H or —CH₃;
R* and R** are independently, H, OH, —OR⁵, or optionally substituted C₁-C₁₂ alkyl;
R⁵ is optionally substituted C₁-C₆ alkyl, or —C(O)NR^{o}R^{o};
X¹ is O or CH wherein if X¹ is O, then there is a single bond between X¹ and X² and a double bond between X² and X³; and wherein X¹ is CH then there is a double bond between X¹ and X² and a single bond between X² and X³;
X² is C; and
X³ is CH or CH₂;
or a pharmaceutically acceptable salt thereof.

2. The compound according to claim 1, having the structure of formula (II) or formula (III): 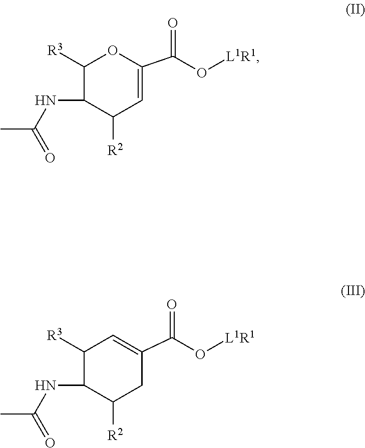
or a pharmaceutically acceptable salt thereof.

3. The compound according to claim 1, wherein R* and R** are independently, H, —OH, —OR⁵, or C₁-C₁₂ alkyl optionally substituted with one or more substituent each independently selected from —OH, —OR⁵, or —OC(O)(C₁-C₈ alkyl).

4. The compound according to claim 1, wherein R³ is —CH(OR⁵)CH(OR⁵)CH₂(OR⁵), —CH(OCH₃)CH₂(OH)CH₂OC(O)(CH₂)₆CH₃.

5. The compound according to claim 1, wherein R² is —NHC(NH₂)NH.

6. The compound according to claim 1, wherein R¹ is —C(O)(CR^{o}R^{o})ᵣC(R^{o}R′)(CR^{o}R^{o})ₛNH₂; and r and s are each independently zero, one or two.

7. The compound according to claim 6, wherein R¹ is —C(O)C(R^{o}R′)NH₂.

8. The compound according to claim 1, wherein L¹ is —C(R⁴)₂O—.

9. The compound according to claim 1, wherein and R′, R″ and R′″ are each independently H, —CH₃, —CH(CH₃)CH₂CH₃, —CH₂CH(CH₃)₂, —CH₂OH, —CH(CH₃)₂, —CH₂C(O)OH, —CH₂CH₂C(O)OH, —CH₂CH₂CH₂CH₂NH₂, —CH₂CH₂CH₂NHC(═NH)NH₂, —CH₂SH, —CH₂C(O)NH₂, —CH₂CH₂C(O)NH₂, —CH(OH)CH₃, —CH₂CH₂SCH₃, 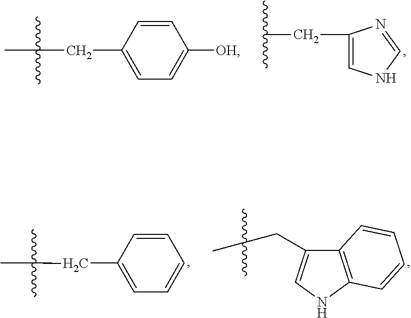
or the side chain that is comprised by proline.

10. The compound according to claim 9, wherein and R′, R″ and R″ are each independently H, —CH₃, —CH(CH₃)CH₂CH₃, —CH₂CH(CH₃)₂, CH(CH₃)₂—CH₂OH, or —CH₂CH₂CH₂CH₂NH₂.

11. The compound according to claim 1, wherein each occurrence of R⁴ is independently H or a C₁-C₆ alkyl group, wherein if one occurrence of R⁴ is H, then the other occurrence of R⁴ is not H or CH₃.

12. The compound according to claim 1, wherein each occurrence of R⁴ is independently H, —CH₃, —CH₂CH₃, —CH₂CH₂CH₃, —CH(CH₃)₂, —CH₂CH₂CH₂CH₃, —CH(CH₃)CH₂CH₃, —CH₂CH(CH₃)₂, —C(CH₃)₃, —CH₂(CH₂)₃CH₃, —CH(CH₃)(CH₂)₂CH₃, —CH(CH₃)CH(CH₃)₂, —C(CH₃)₂CH₂CH₃, —CH(CH₂CH₃)₂, —CH₂CH(CH₃)CH₂CH₃, —CH₂CH₂CH(CH₃)₂, —CH₂C(CH₃)₃, CH₂(CH₂)CH₃, —CH(CH₃)(CH₂)₃CH₃, —CH₂CH(CH₃)(CH₂)₂CH₃, —CH₂CH₂CH(CH₃)CH₂CH₃, —CH₂(CH₂)₂CH(CH₃)₂, —CH(CH₃)CH(CH₃)CH₂CH₃, —CH(CH₃)CH₂CH(CH₃)₂, —CH₂CH(CH₃)CH(CH₃)₂, —CH₂CH₂CH₂CH(CH₃)₂, —C(CH₃)₂CH₂CH₂CH₃, —C(CH₃)₂CH(CH₃)₂, —C(CH₂CH₃)₂CH₃, or —CH₂CH(CH₂CH₃)₂, 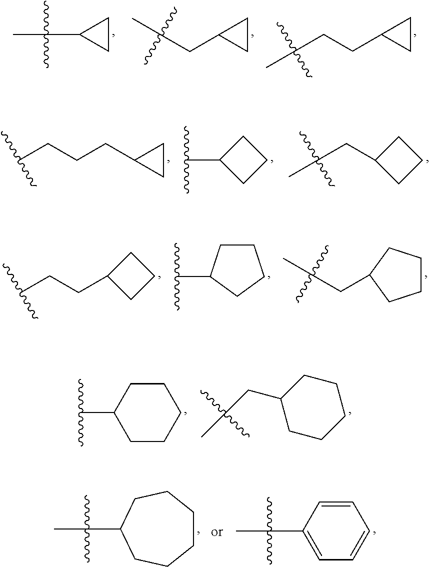
wherein if one occurrence of R⁴ is H, then the other occurrence of R⁴ is not H or CH₃.

13. The compound according to claim 2, wherein L¹ is —CH(CH(CH₃)₂)O—, R¹ is —C(O)CH(C(CH₃)₂)NH₂, R² is —NHC(NH₂)NH and R³ is —CH(OH)CH(OH)CH₂(OH); L¹ is —CH(CH(CH₃)₂)O—, R¹ is —C(O)CH(C(CH₃)₂)NH₂, R² is —NHC(NH₂)NH and R³ is —CH(OCH₃)CH(OH)CH₂(OH); L¹ is —CH(CH(CH₃)₂)O—, R¹ is —C(O)CH(C(CH₃)₂)NH₂, R² is —NHC(NH₂)NH and R³ is —CH(OCH₃)CH₂(OH)CH₂OC(O)(CH₂)₆CH₃; or L¹ is —CH(CH(CH₃)₂)O—, R¹ is —C(O)CH(C(CH₃)₂)NH₂, R² is —NHC(NH₂)NH and R³ is —OCH(CH₂CH₃)₂.

14. A compound which is:

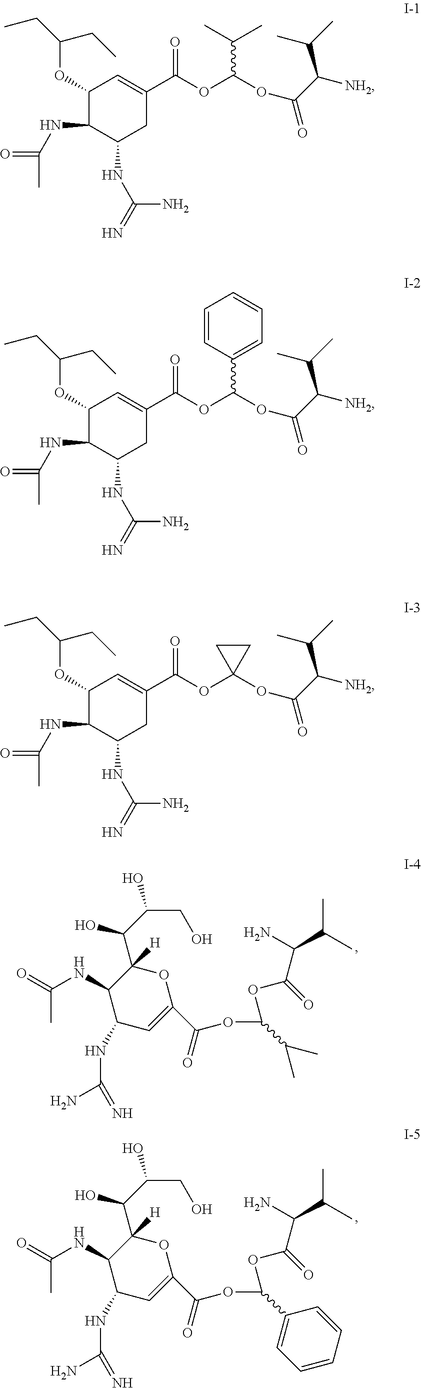

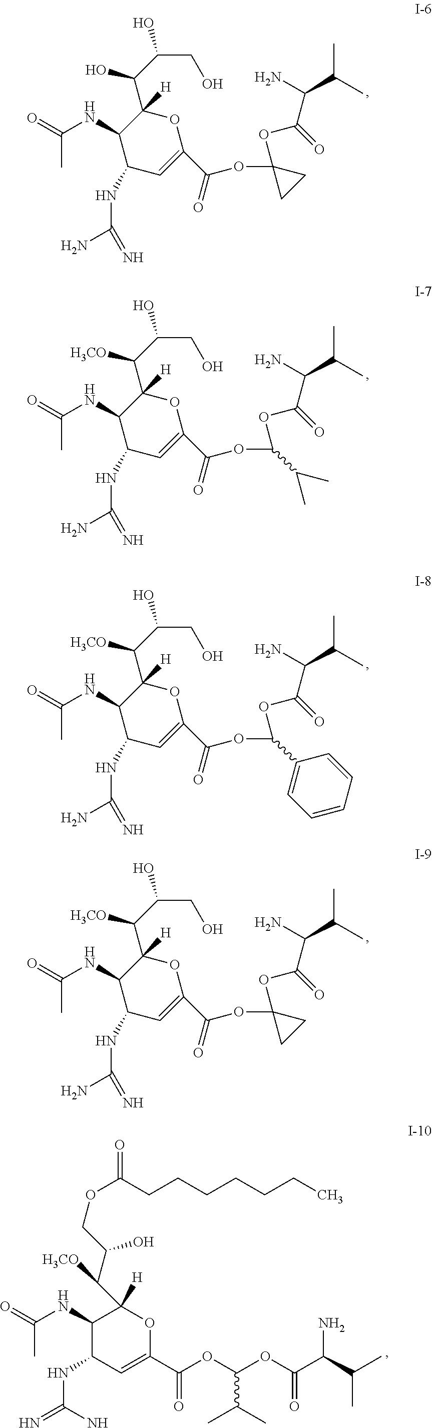

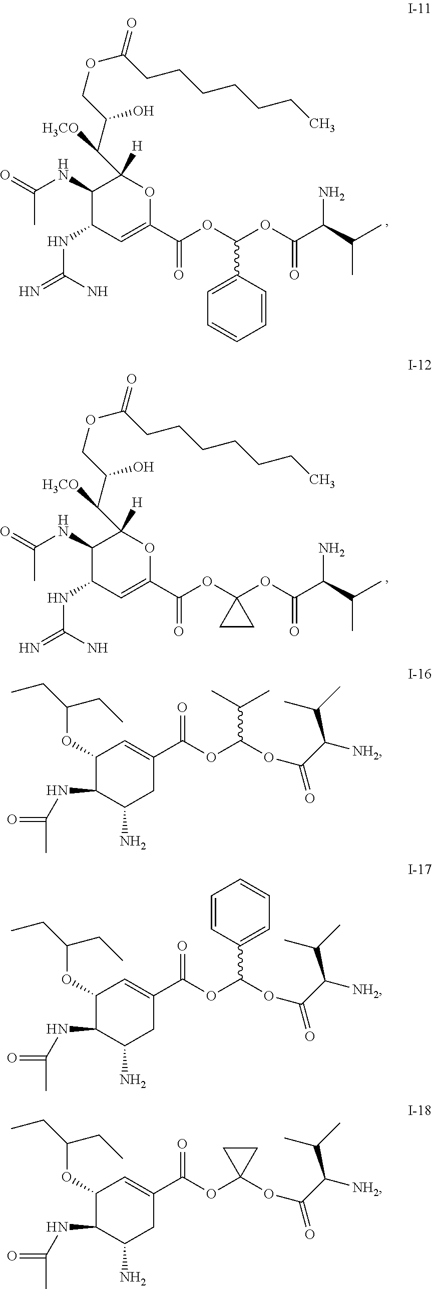

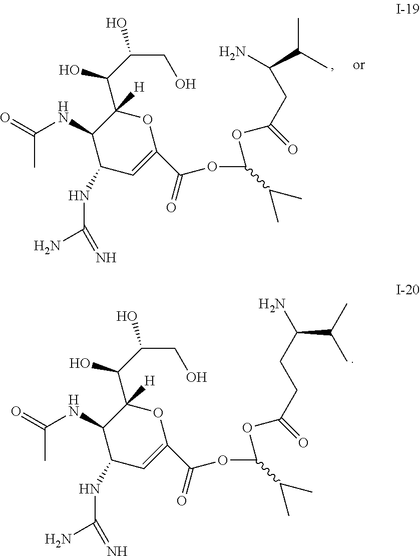 [SMILES: CC(=O)N[C@H]1[C@H]([C@@H](O)[C@H](O)CO)OC(C(=O)OC(OC(=O)C[C@@H](N)C(C)C)C(C)C)=C[C@@H]1NC(=N)N ; CC(=O)N[C@H]1[C@H]([C@@H](O)[C@H](O)CO)OC(C(=O)OC(OC(=O)CC[C@@H](N)C(C)C)C(C)C)=C[C@@H]1NC(=N)N]

15. A pharmaceutical composition comprising a compound according to claim 1.

16. A method of treating a viral infection in a subject, comprising: administering a compound according to claim 1, to a subject in need thereof.

17. The method according to claim 16, wherein the viral infection is an influenza virus infection.

18. A compound of formula (IV): 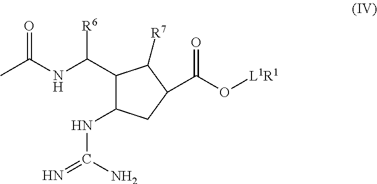
wherein: L¹ is —(CR^{o}R^{o})ₘC(R⁴)₂(CR^{o}R^{o})ₙO(CR^{o}R^{o})ₒ—;
R¹ is —C(O)(CR^{o}R^{o})ᵣC(R^{o}R′)(CR^{o}R^{o})ₛNH₂, —C(O)(CR^{o}R^{o})ᵣC(R^{o}R′)(CR^{o}R^{o})ₛN(H)C(O)(CR^{o}R^{o})_{w}C(R^{o}R″)(CR^{o}R^{o})ₓNH₂, or —C(O)(CR^{o}R^{o})ᵣC(R^{o}R′)(CR^{o}R^{o})ₛN(H)C(O)(CR^{o}R^{o})_{w}C(R^{o}R″)(CR^{o}R^{o})ₓN(H)C(O)(CR^{o}R^{o})_{y}C(R^{o}R″′)(CR^{o}R^{o})_{z}NH₂;
each occurrence of m, n, o, r, s, w, x, y, or z is independently zero, one, or two;
each occurrence of R^{o} is independently alkyl, substituted alkyl, cycloalkyl, substituted cycloalkyl, aryl, substituted aryl, heteroaryl and substituted heteroaryl;
each occurrence of R⁴ is independently hydrogen or an optionally substituted group selected from a C₁-C₆ alkyl group, a 3-7 membered saturated, partially saturated or fully unsaturated monocyclic ring having 0-3 heteroatoms independently selected from nitrogen or oxygen or two occurrences of R⁴ are taken together with the atom(s) to which they are bound to form an optionally substituted 3-7 membered ring, wherein if one occurrence of R⁴ is H, then the other occurrence of R⁴ is not H or CH₃;
R⁵ is optionally substituted C₁-C₄ alkyl, —C(O)NR^{o}R^{o};
R⁶ is C₁-C₁₀ alkyl; and
R⁷ is —OH, —OR⁵, C₁-C₆ alkyl or —NR^{o}R^{o};
or a pharmaceutically acceptable salt thereof.

19. The compound according to claim 18, wherein R⁶ is —C(CH₂CH₃)₂.

20. The compound according to claim 18, wherein R⁷ is —OH or —OR⁵.
